(19) United States
(12) Patent Application Publication
(10) Pub. No.: US 2025/0375458 A1
(43) Pub. Date: Dec. 11, 2025

(54) COMPOSITIONS AND METHODS TO COMBAT MULTIDRUG-RESISTANT AND PERSISTENT T-CELL-MEDIATED, ONCOLOGICAL AND INFECTIOUS DISEASES

(71) Applicant: Theralase Technologies, Inc., Toronto (CA)

(72) Inventors: Arkady Mandel, North York (CA); Roger Dumoulin-White, Toronto (CA)

(21) Appl. No.: 18/734,622

(22) Filed: Jun. 5, 2024

(51) Int. Cl. A61K 31/555; A61K 38/40; A61K 45/06; A61P 35/00

(52) U.S. Cl. CPC A61K 31/555; A61K 38/40; A61K 45/06; A61P 35/00

(57) ABSTRACT

Disclosed is a method for treating a condition, which includes the steps of: selecting a patient for treatment who has the condition; administering to the patient at least one active pharmaceutical ingredient (API); and administering to the patient at least one metal complex represented by formula (I):

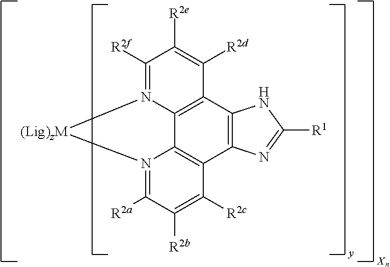

including hydrates, solvates, pharmaceutically acceptable salts and prodrugs thereof. Also disclosed is a composition for conducting the method, which includes: at least one API selected from the group consisting of Cisplatin, Temozolomide, Vandetanib, Withaferin A, Vemurafenib, Amiodarone, Vinblastine, Metformin, Gemcitabine and Acyclovir; and at least one metal complex effective to potentiate an efficacy of the API to treat the condition, wherein the at least one metal complex is represented by formula (I), including hydrates, solvates, pharmaceutically acceptable salts and prodrugs thereof.

### BACKGROUND OF THE INVENTION

### 1. Field of Invention

This invention relates to complexes and methods for their use in the prevention or treatment of resistance to therapies for treating T-cell mediated, oncological and/or infectious discases.

### 2. Description of Related Art

As overactivated T-cells lead to clinical symptoms of immune-mediated and/or autoimmune-mediated diseases, dysfunctional T-cells can lead to the development of infectious diseases and cancer.

T-cells are crucial for immune functions to maintain health and prevent discase. T-cell development occurs in a stepwise process in the thymus and mainly generates CD4⁺ and CD8⁺T-cell subsets. Upon antigen stimulation, naïve T-cells differentiate into CD4⁺ helper and CD8⁺ cytotoxic effector and memory cells, mediating direct killing, diverse immune regulatory function and long-term protection. In response to acute and chronic infections and tumors, T-cells adopt distinct differentiation trajectories and develop into a range of heterogeneous populations with various phenotype, differentiation potential and functionality under precise and elaborate regulations of transcriptional and epigenetic programs. Abnormal T-cell immunity can initiate and promote the pathogenesis of autoimmune diseases.

Cancer treatment has reached promising breakthroughs during the last decades.

Chemotherapy is currently among the principal modes of treatment for cancer patients. Clinically, many tumors present a satisfactory response when they are first exposed to chemotherapeutic drugs.

Likewise, many infectious diseases are routinely treated with a variety of recently developed antiviral and antibacterial treatments.

Despite the success of these treatments, drug-resistance has become a hurdle to achieve long-term positive results in treating diseased cells.

Cancer can become resistant to many different types of drugs. Increased efflux of drugs, enhanced repair/increased tolerance to DNA damage, high antiapoptotic potential, decreased permeability and enzymatic deactivation allow cancer cells to survive chemotherapy.

Cancers develop resistance to treatments such as chemotherapy, radiotherapy and other targeted therapies through many different mechanisms. These include specific genetic and epigenetic changes in the cancer cell and/or the microenvironment in which the cancer cell resides.

This resistance to standard therapy, including drugs, biologics, chemotherapy, gene therapy, radiation therapy and/or other methods of treatment, is a common occurrence resulting in the loss of therapeutic response, despite the wide spectrum of drugs and treatments available.

The treatment of acute and chronic diseases, including infectious diseases and cancers, with antibiotics, antimicrobial and/or antiviral drugs, as well as chemotherapeutic drugs and/or radiation therapy is frequently impaired, ineffective or quickly wanes in effectiveness because of drug and/or immune and/or radiation therapy resistance. In these cases, infected cancer cells and/or tumor tissues can be resistant to a variety of therapeutic approaches; including, drugs with different structures and mechanisms of action. This phenomenon is termed multidrug resistance (MDR). See, e.g., Catalano et al. Multidrug Resistance (MDR): A Widespread Phenomenon in Pharmacological Therapies. Molecules. 2022 Jan. 18; 27(3):616. doi: 10.3390/molecules27030616. PMID: 35163878; PMCID: PMC8839222.

Treatment resistance is a complex process that arises from alterations in the therapeutic targets. For example, cancer cell resistance against anticancer agents can be due to many factors, one of which is the individual's genetic differences, especially in tumoral somatic cells.

Drug resistance can arise via different mechanisms, including: multi-drug resistance, cell death inhibition (apoptosis suppression), altering of the cell metabolism, epigenetics, how the drug targets the cell, enhanced DNA repair and gene amplification.

Although there are several different mechanisms associated with the development of MDR, a common cause is believed to be overexpression of a plasma membrane glycoprotein; specifically, an MDRI gene product. This MDRI gene product belongs to the ABC (ATP binding cassette) superfamily of transporter proteins, and it acts as an energy-dependent drug efflux pump, preventing adequate intracellular accumulation of a broad range of cytotoxic drugs including anthracyclines (i.e.: doxorubicin, daunorubicin), vinca alkaloids (i.e.: vincristine, vinblastine), taxanes (i.e.: paclitaxel, docetaxel) and many others for cell kill; therefore, this underlines the critical importance of identifying compounds that could inhibit the efflux pumps activities.

Moreover, efflux pumps play a major role in increasing non-cancerous disease resistance; hence, rendering many drugs of little use. For example, large numbers of pathogens are becoming multidrug resistant due to inadequate dosage and use of existing antimicrobials. This leads to the need for identifying new efflux pump inhibitors. Design of novel targeted therapies using inherent complexities of the biological network model has gained increasing importance in recent times. See Huang et al. Bacterial Multidrug Efflux Pumps at the Frontline of Antimicrobial Resistance: An Overview. Antibiotics (Basel). 2022 Apr. 13; 11(4):520. doi: 10.3390/antibiotics 11040520. PMID: 35453271; PMCID: PMC9032748.

Thus, MDR is common in the treatment of tumors and infectious diseases. As a result of the lack of efficacy of treatment, the majority of patients progress in their disease. The mechanisms of treatment failure of therapeutic drugs have been well studied. For example, via a unique protection system (i.e., MDR), cancer cells can escape the toxic effect of many drugs in spite of their different chemical structures and different mechanisms of intracellular activity.

It is therefore desired to provide compounds, compositions and therapeutic methods for treating cancer, in which the MDR effect is reduced or preferably eliminated.

It is further desired to provide compounds, compositions and therapeutic methods for treating infectious diseases, in which the MDR effect is reduced or preferably eliminated.

It is still further desired to provide synergistic interventions that significantly increase potency and/or efficacy of prescribed therapies, offering the opportunity for more precise control biological system, and leading to a long-lasting or permanent remission based on regained immune balance and self-tolerance.

All references cited herein are incorporated herein by reference in their entireties. The citation of any reference is not to be construed as an admission that it is prior art with respect to the present invention.

### BRIEF SUMMARY OF THE INVENTION

Accordingly, a first aspect of the invention is a method for treating a condition, said method comprising the steps of: selecting a patient for treatment who has the condition; administering to the patient at least one active pharmaceutical ingredient (API); and administering to the patient at least one metal complex represented by formula (I):

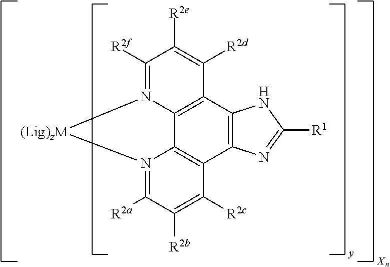

including hydrates, solvates, pharmaceutically acceptable salts and prodrugs thereof, wherein:
M is selected from the group consisting of manganese, molybdenum, rhenium, iron, ruthenium, osmium, cobalt, rhodium, iridium, nickel, platinum, and copper;
X is selected from the group consisting of Cl⁻, PF₆⁻, Br⁻, BF₄⁻, ClO₄⁻, CF₃SO₃⁻, and SO₄⁻².
n=0, 1, 2, 3, 4, or 5;
y=1, 2, or 3;
z=0, 1, or 2;
Lig at each occurrence is independently selected from the group consisting of

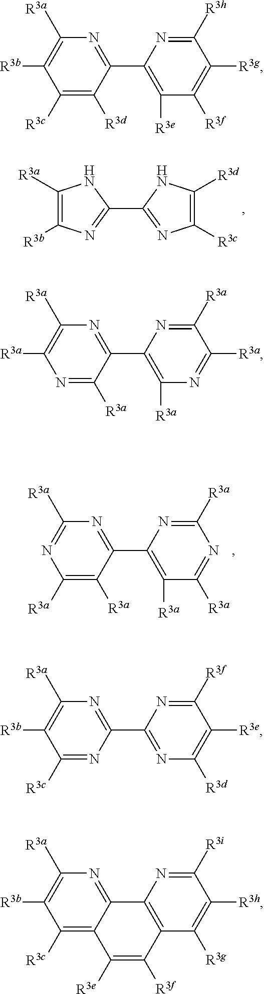

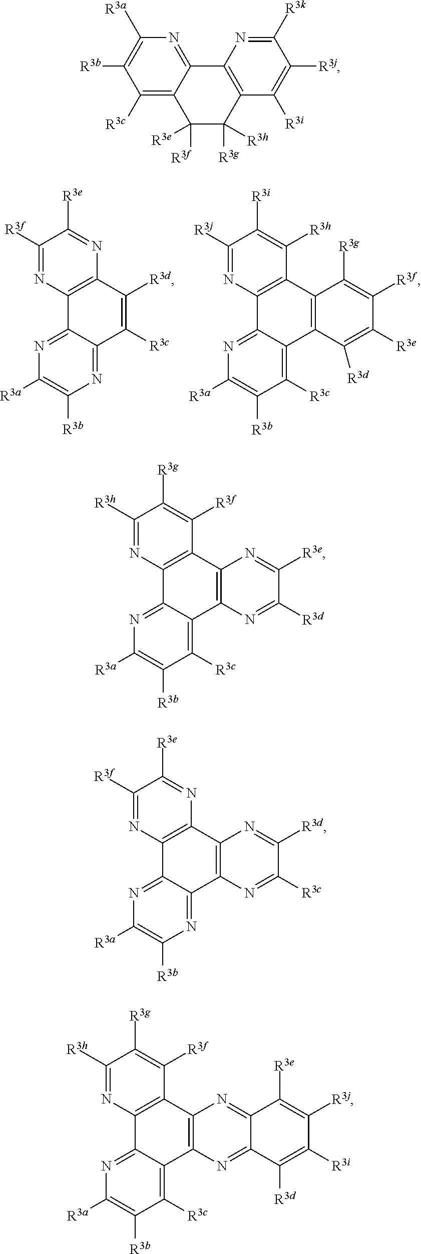

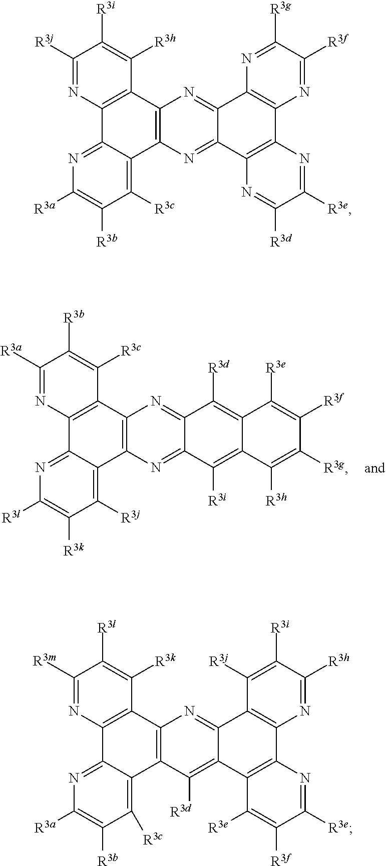

R¹ is selected from the group consisting of

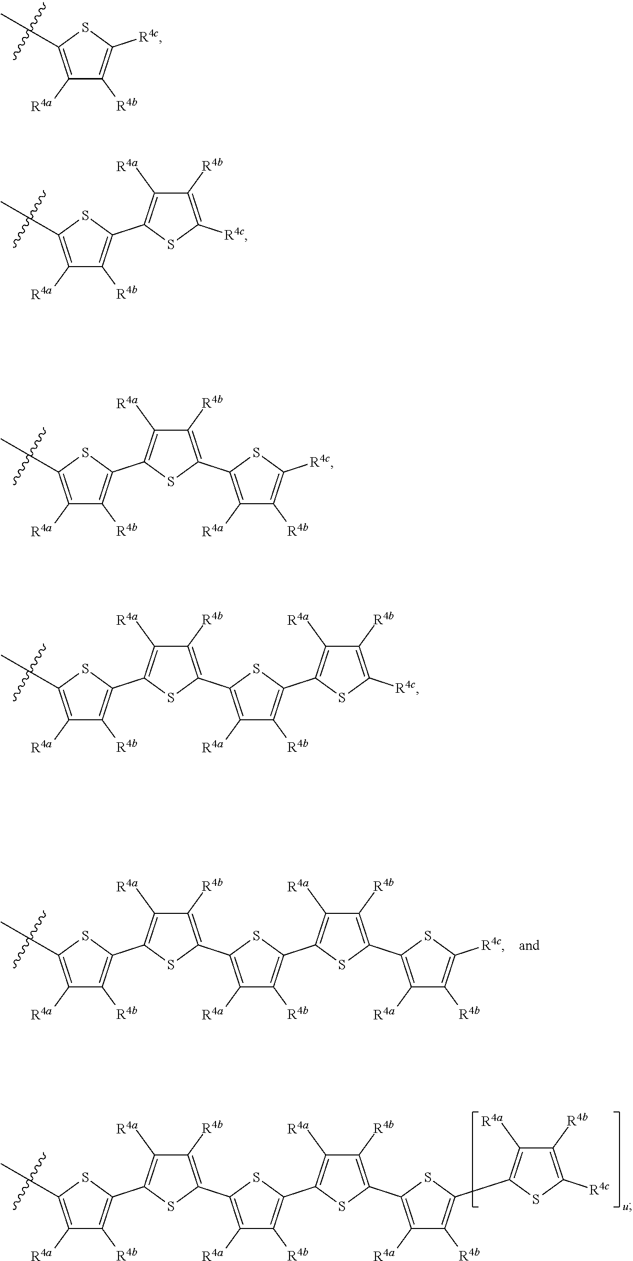

u is an integer of 1 to 20;
R^{2a}, R^{2b}, R^{2c}, R^{2d}, R^{2e}, and R^{2f} at each occurrence are each independently selected from the group consisting of hydrogen, C1-6 optionally substituted alkyl, C1-6 optionally substituted branched alkyl, C3-7 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h} R³ⁱ, R^{3j}, R^{3k}, R^{3l}, and R^{3m} at each occurrence are each independently selected from the group consisting of hydrogen, C1-6 optionally substituted alkyl, C1-6 optionally substituted branched alkyl, C1-6 optionally substituted haloalkyl, C1-6 optionally substituted alkoxy, and CO₂R⁸;
R^{4a}, R^{4b}, and R^{4c} at each occurrence are each independently selected from the group consisting of hydrogen, C1-6 optionally substituted alkyl, C1-6 optionally substituted branched alkyl, C1-6 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence is independently selected from the group consisting of hydrogen and optionally substituted alkyl;
R⁶ at each occurrence is independently selected from the group consisting of hydrogen and optionally substituted alkyl;
R⁷ at each occurrence is independently selected from the group consisting of hydrogen and optionally substituted alkyl; and
R⁸ at each occurrence is independently selected from the group consisting of hydrogen and optionally substituted alkyl,
wherein:
the administering to the patient of the at least one metal complex is conducted prior to, during and/or after the administering to the patient of the at least one API; and
the at least one API has a structure not represented by Formula (I).

In certain embodiments, the administering to the patient of the at least one metal complex causes an improvement in an efficacy of treating the condition relative to continued treatment of the condition with only the at least one API.

In certain embodiments, the at least one metal complex is administered to the patient only after the condition has developed a resistance to the at least one API, which continues to be administered to the patient along with the at least one metal complex.

In certain embodiments, the at least one metal complex is administered to the patient prior to or concurrently with the step of administering to the patient the at least one active pharmaceutical ingredient.

In certain embodiments, the condition is cancer, and the at least one API is at least one member selected from the group consisting of a chemotherapeutic agent, an immunotherapeutic agent and a targeted therapeutic agent.

In certain embodiments, the condition is an infectious disease and the at least one API is at least one member selected from the group consisting of an antibiotic agent, an antimicrobial agent, an antifungal agent and an antiviral agent.

In certain embodiments, the at least one API is selected from the group consisting of Cisplatin, Temozolomide, Vandetanib, Withaferin A, Vemurafenib, Amiodarone, Vinblastine, Metformin, Gemcitabine and Acyclovir.

In certain embodiments, the method further comprises administering transferrin to the patient.

In certain embodiments, the method further comprises administering to the patient radiation selected from the group consisting of infrared light, visible light, X-rays and gamma rays.

In certain embodiments, the at least one metal complex comprises at least one member selected from the group consisting of:
Ru(2,2′-bipyridine)₂(2-(2′,2″:5″,2′″-terthiophene)-imidazo[4,5-f][1,10] phenanthroline);
Ru(2,2′-bipyridine)₂(2-(2′,2″:5″,2″′;5″′,2″″-quaterthiophene)-imidazo[4,5-f][1,10] phenanthroline);
Ru(4,4′-dimethyl-2,2′-bipyridine)₂(2-thiophenimidazo[4,5-f][1,10] phenanthroline);
Ru(4,4′-dimethyl-2,2′-bipyridine)₂(2-(2′,2″-bithiophene)-imidazo[4,5-f][1,10] phenanthroline);
Ru(4,4′-dimethyl-2,2′-bipyridine)₂(2-(2′,2″:5″,2′″-terthiophene)-imidazo[4,5-f][1,10] phenanthroline);
Ru(4,4′-dimethyl-2,2′-bipyridine)₂(2-(2′,2″:5″,2″′;5″′,2″″-quaterthiophene) -imidazo[4,5-f][1,10] phenanthroline);
Ru(4,4′-di-t-butyl-2,2′-bipyridine)₂(2-thiophenimidazo[4,5-f][1,10] phenanthroline);
Ru(4,4′-di-t-butyl-2,2′-bipyridine)₂(2-(2′,2″-bithiophene)-imidazo[4,5-f][1,10] phenanthroline);
Ru(4,4′-di-t-butyl-2,2′-bipyridine)₂(2-(2′,2″:5″,2′″-terthiophene)-imidazo[4,5-f][1,10] phenanthroline);
Ru(4,4′-dimethoxy-2,2′-bipyridine)₂(2-(2′,2″:5″,2″′-terthiophene)-imidazo[4,5-f][1,10] phenanthroline);
Ru(5,5′-dimethyl-2,2′-bipyridine)₂(2-thiophenimidazo[4,5-f][1,10] phenanthroline);
Ru(5,5′-dimethyl-2,2′-bipyridine)₂(2-(2′,2″-bithiophene)-imidazo[4,5-f][1,10] phenanthroline);
Ru(5,5′-dimethyl-2,2′-bipyridine)₂(2-(2′,2″:5″,2′″-terthiophene)-imidazo[4,5-f][1,10] phenanthroline);
Ru(6,6′-dimethyl-2,2′-bipyridine)₂(2-thiophenimidazo[4,5-f][1,10] phenanthroline);
Ru(6,6′-dimethyl-2,2′-bipyridine)₂(2-(2′,2″-bithiophene)-imidazo[4,5-f][1,10] phenanthroline);
Ru(6,6′-dimethyl-2,2′-bipyridine)₂(2-(2′,2″:5″,2″′-terthiophene)-imidazo[4,5-f][1,10] phenanthroline);
Ru(4,4′-di (methylcarboxy)-2,2′-bipyridine)₂(2-(2′,2″:5″,2′″-terthiophene) -imidazo[4,5-f][1,10] phenanthroline);
Ru(2,2′-bipyrimidine)₂(2-(2′,2″:5″,2″′-terthiophene)-imidazo[4,5-f][1,10] phenanthroline);
Ru(2,2′-bipyrimidine)(4,4′-dimethyl-2,2′-bipyridine)(2-(2′,2″:5″,2″′-terthiophene)-imidazo[4,5-f][1,10] phenanthroline);
Ru(1,10-phenanthroline)₂(2-thiophenimidazo[4,5-f][1,10] phenanthroline);
Os(2,2′-bipyridine)₂(2-thiophenimidazo[4,5-f][1,10] phenanthroline);
Os(2,2′-bipyridine)₂(2-(2′,2″-bithiophene)-imidazo[4,5-f][1,10] phenanthroline);
Os(2,2′-bipyridine)₂(2-(2′,2″:5″,2′″-terthiophene)-imidazo[4,5-f][1,10] phenanthroline);
Os(1,10-phenanthroline)₂(2-thiophenimidazo[4,5-f][1,10] phenanthroline);
Os(1,10-phenanthroline)₂(2-(2′,2″-bithiophene)-imidazo[4,5-f][1,10] phenanthroline);
Os(1,10-phenanthroline)₂(2-(2′,2″:5″,2″′-terthiophene)-imidazo[4,5-f][1,10] phenanthroline);
Os(4,4′-dimethyl-2,2′-bipyridine)₂(2-thiophenimidazo[4,5-f][1,10] phenanthroline);
Os(4,4′-dimethyl-2,2′-bipyridine)₂(2-(2′,2″-bithiophene)-imidazo[4,5-f][1,10] phenanthroline);
Os(4,4′-dimethyl-2,2′-bipyridine)₂(2-(2′,2″:5″,2′″-terthiophene)-imidazo[4,5-f][1,10] phenanthroline); and
pharmaceutically acceptable salts thereof.

In certain embodiments, the at least one metal complex is Ru(4,4′-dimethyl-2,2′-bipyridine)₂(2-(2′,2″:5″,2′″-terthiophene)-imidazo[4,5-f][1,10] phenanthroline) or a pharmaceutically acceptable salt thereof.

A second aspect of the invention is a composition for combination therapy effective to treat a condition in a patient, said composition comprising: at least one active pharmaceutical ingredient (API) selected from the group consisting of: Cisplatin, Temozolomide, Vandetanib, Withaferin A, Vemurafenib, Amiodarone, Vinblastine, Metformin, Gemcitabine and Acyclovir; and at least one metal complex effective to potentiate an efficacy of the active pharmaceutical ingredient to treat the condition, wherein the at least one metal complex is represented by formula (I) as defined above, including hydrates, solvates, pharmaceutically acceptable salts and prodrugs thereof.

In certain embodiments of the composition, the condition is cancer.

In certain embodiments of the composition, the condition is an infectious disease.

In certain embodiments, the composition further comprises transferrin.

In certain embodiments of the composition, the at least one metal complex comprises at least one member selected from the group consisting of:
Ru(2,2′-bipyridine)₂(2-(2′,2″:5″,2′″-terthiophene)-imidazo[4,5-f][1,10] phenanthroline);
Ru(2,2′-bipyridine)₂(2-(2′,2″:5″,2″′;5′″,2″″-quaterthiophene)-imidazo[4,5-f][1,10] phenanthroline);
Ru(4,4′-dimethyl-2,2′-bipyridine)₂(2-thiophenimidazo[4,5-f][1,10] phenanthroline);
Ru(4,4′-dimethyl-2,2′-bipyridine)₂(2-(2′,2″-bithiophene)-imidazo[4,5-f][1,10] phenanthroline);
Ru(4,4′-dimethyl-2,2′-bipyridine)₂(2-(2′,2″:5″,2′″-terthiophene)-imidazo[4,5-f][1,10] phenanthroline);
Ru(4,4′-dimethyl-2,2′-bipyridine)₂(2-(2′,2″:5″,2″′;5″′,2″″-quaterthiophene) -imidazo[4,5-f][1,10] phenanthroline);
Ru(4,4′-di-t-butyl-2,2′-bipyridine)₂(2-thiophenimidazo[4,5-f][1,10] phenanthroline);
Ru(4,4′-di-t-butyl-2,2′-bipyridine)₂(2-(2′,2″-bithiophene)-imidazo[4,5-f][1,10] phenanthroline);
Ru(4,4′-di-t-butyl-2,2′-bipyridine)₂(2-(2′,2″:5″,2′″-terthiophene)-imidazo[4,5-f][1,10] phenanthroline);
Ru(4,4′-dimethoxy-2,2′-bipyridine)₂(2-(2′,2″:5″,2″′-terthiophene)-imidazo[4,5-f][1,10] phenanthroline);
Ru(5,5′-dimethyl-2,2′-bipyridine)₂(2-thiophenimidazo[4,5-f][1,10] phenanthroline);
Ru(5,5′-dimethyl-2,2′-bipyridine)₂(2-(2′,2″-bithiophene)-imidazo[4,5-f][1,10] phenanthroline);
Ru(5,5′-dimethyl-2,2′-bipyridine)₂(2-(2′,2″:5″,2′″-terthiophene)-imidazo[4,5-f][1,10] phenanthroline);
Ru(6,6′-dimethyl-2,2′-bipyridine)₂(2-thiophenimidazo[4,5-f][1,10] phenanthroline);
Ru(6,6′-dimethyl-2,2′-bipyridine)₂(2-(2′,2″-bithiophene)-imidazo[4,5-f][1,10] phenanthroline);
Ru(6,6′-dimethyl-2,2′-bipyridine)₂(2-(2′,2″:5″,2′″-terthiophene)-imidazo[4,5-f][1,10] phenanthroline);
Ru(4,4′-di (methylcarboxy)-2,2′-bipyridine)₂(2-(2′,2″:5″,2′″-terthiophene) -imidazo[4,5-f][1,10] phenanthroline);
Ru(2,2′-bipyrimidine)₂(2-(2′,2″:5″,2″′-terthiophene)-imidazo[4,5-f][1,10] phenanthroline);
Ru(2,2′-bipyrimidine)(4,4′-dimethyl-2,2′-bipyridine)(2-(2′,2″:5″,2′″-terthiophene)-imidazo[4,5-f][1,10] phenanthroline);
Ru(1,10-phenanthroline)₂(2-thiophenimidazo[4,5-f][1,10] phenanthroline);
Os(2,2′-bipyridine)₂(2-thiophenimidazo[4,5-f][1,10] phenanthroline);
Os(2,2′-bipyridine)₂(2-(2′,2″-bithiophene)-imidazo[4,5-f][1,10] phenanthroline);
Os(2,2′-bipyridine)₂(2-(2′,2″:5″,2′″-terthiophene)-imidazo[4,5-f][1,10] phenanthroline);
Os(1,10-phenanthroline)₂(2-thiophenimidazo[4,5-f][1,10] phenanthroline);
Os(1,10-phenanthroline)₂(2-(2′,2″-bithiophene)-imidazo[4,5-f][1,10] phenanthroline);
Os(1,10-phenanthroline)₂(2-(2′,2″:5″,2′″-terthiophene)-imidazo[4,5-f][1,10] phenanthroline);
Os(4,4′-dimethyl-2,2′-bipyridine)₂(2-thiophenimidazo[4,5-f][1,10] phenanthroline);
Os(4,4′-dimethyl-2,2′-bipyridine)₂(2-(2′,2″-bithiophene)-imidazo[4,5-f][1,10] phenanthroline);
Os(4,4′-dimethyl-2,2′-bipyridine)₂(2-(2′,2″:5″,2′″-terthiophene)-imidazo[4,5-f][1,10] phenanthroline); and pharmaceutically acceptable salts thereof.

In certain embodiments of the composition, the metal complex is Ru(4,4′-dimethyl-2,2′-bipyridine)₂(2-(2′,2″:5″,2′-terthiophene)-imidazo[4,5-f][1,10] phenanthroline) or a pharmaceutically acceptable salt thereof.

Other features and advantages of the present invention will become apparent from the following detailed description, examples and figures. It should be understood; however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF SEVERAL VIEWS OF THE DRAWINGS

The invention will be described in conjunction with the following drawings, wherein:

FIG. 1A is a graph of surviving fraction of U251 human glioma cells as a function of temozolomide concentration.

FIG. 1B is a bar graph of cell kill percentage of U87 human glioma cells treated with temozolomide (TMZ), treated with RUTHERRIN, treated with both or treated with neither.

FIG. 2 is a bar graph of cell kill percentage of U87 human glioma cells treated with different chemotherapeutic drugs with and without RUTHERRIN.

FIG. 3A is a graph of percent survival against time over 45 days.

FIG. 3B is a graph of tumor volume against time over 30 days.

FIG. 4A is a bar graph of median fluorescence intensity (MFI) of A549 Human NSCLC cells treated with RUVIDAR, RUTHERRIN or neither.

FIG. 4B is a bar graph of MFI of U87 human glioma cells treated with RUVIDAR, RUTHERRIN or neither.

FIG. 4C is a bar graph of relative TMZ retention of U87 human glioma cells treated with RUTHERRIN or untreated.

FIG. 5 is a bar graph of cell kill percent of T24 human bladder cancer cells treated with metformin alone, metformin plus RUVIDAR or metformin plus RUTHERRIN.

FIG. 6 is a bar graph of cell kill percent of U87 human glioma cells treated with three different doses of RUVIDAR in the presence or absence of N-acetylcysteine.

FIG. 7A is a bar graph showing the yield suppression of HSV-1 propagated in host cells that were untreated, treated with RUVIDAR and/or Acyclovir, or treated with DMSO and/or RUVIDAR.

FIG. 7A is a bar graph showing the yield suppression of HSV-1 propagated in host cells that were untreated, treated with RUTHERRIN and/or Acyclovir, or treated with DMSO and/or RUTHERRIN.

FIG. 8A is a bar graph of the percentage change in expression of CD47 and PDL-1 in T24 human bladder cancer cells with or without RUVIDAR treatment.

FIG. 8B is a bar graph of the percentage change in expression of CD47 and PDL-1 in U87 human glioma cells with or without RUTHERRIN treatment.

FIG. 8C is a bar graph of the percentage change in CD47 expression in U87 human glioma cells without treatment, or after treatment with RUTHERRIN or photodynamic therapy (PDT) and RUTHERRIN.

FIG. 8D is a bar graph of the percentage change in CD47 expression in A549 human lung cancer cells, which were: untreated, treated only with RUVIDAR, treated only with RUTHERRIN, treated only with ionizing radiation, treated with RUVIDAR and ionizing radiation, or treated with RUTHERRIN and ionizing radiation.

FIG. 9A is a bar graph of relative cisplatin retention with and without RUTHERRIN treatment in non-small cell lung cancer cells.

FIG. 9B is a bar graph of relative gemcitabine retention with and without RUTHERRIN treatment in bladder cancer cells.

FIG. 10A is a bar graph of percentage cell kill with or without RUTHERRIN or cisplatin treatment in lung cancer cells.

FIG. 10B is a bar graph of percentage cell kill with or without RUTHERRIN or gemcitabine treatment in bladder cancer cells.

### DETAILED DESCRIPTION OF PREFERRED EMBODIMENTS OF THE INVENTION

### Glossary

Throughout the description, where compositions are described as: having, including or comprising specific components or where processes are described as: having, including or comprising specific process steps, it is contemplated that compositions of the present teachings also consist essentially of, or consist of, the recited components and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial, so long as the present teachings remain operable. Moreover, two or more steps or actions can be conducted simultaneously.

For the purposes of the present invention the terms “compound”, “complex”, “metal complex” and “composition of matter” stand equally well for the inventive complexes described herein, be they photodynamic or not, including all enantiomeric forms, diastereomeric forms, salts and the like and the terms “compound”, “complex”, “metal complex” and “composition of matter” are used interchangeably throughout this specification.

Compounds described herein can contain an asymmetric atom (also referred as a chiral center), and some of the compounds can contain one or more asymmetric atoms or centers, which can thus give rise to optical isomers (enantiomers) and diastercomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to: diastereomeric salt formation, kinetic resolution and asymmetric synthesis. The present teachings also encompass cis and trans isomers of compounds containing alkenyl moieties (e.g.: alkenes and imines). It is also understood that the present teachings encompass all possible regioisomers, and mixtures thereof, which can be obtained in pure form by standard separation procedures known to those skilled in the art, and include, but are not limited to: column chromatography, thin-layer chromatography and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include: metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di-or tri-lower alkylamine (e.g.: ethyl-tert-butyl-, diethyl-, diisopropyl-, tricthyl-, tributyl-or dimethylpropylamine), or a mono-, di-, or trihydroxy lower alkylamine (e.g.: mono-, di-or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic, as well as other known pharmaceutically acceptable acids.

When any variable occurs more than one time in any constituent or in any formula, its definition in each occurrence is independent of its definition at every other occurrence (e.g.: in N(R⁶)₂, each R⁶ may be the same or different than the other). Combinations of substituents and/or variables are permissible only if such combinations result in stable compounds.

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

As used herein, the term “photodynamic therapy” or “PDT” refers to a treatment for destroying cells or modulating immune function, including immune response, of cells and tissue through use of a drug that can be activated by light of a certain wavelength and dose.

As used herein, the term “radiodynamic therapy” or “RDT” refers to a treatment for destroying cells or modulating immune function, including immune response, of cells and tissue through use of a drug that can be activated by ionizing radiation of a certain wavelength and dose.

As used herein, the term “photodynamic compound” or “PDC” refers to a compound that can be activated by light of a certain wavelength and dose for PDT. The term is also used herein to refer to a compound that can be activated by ionizing radiation of a certain wavelength and dose for RDT.

As used herein, the term “radiation” used without the term “ionizing” is intended to encompass all types of radiation in the electromagnetic spectrum including light and ionizing radiation.

As used herein, the term “immunotherapy” refers to a treatment which elicits an immune response from a patient so as to prevent, ameliorate or cure a condition (e.g., a disease or an infection).

An “immunotherapeutic agent” is a substance that elicits an immune response from a patient, so as to prevent, ameliorate or cure a condition (e.g.: a disease or an infection).

Except when noted, the terms “subject” or “patient” are used interchangeably and refer to mammals such as human patients and non-human primates, as well as experimental animals such as: dogs, rabbits, rats, mice and other animals. Accordingly, the term “subject” or “patient” as used herein means any mammalian patient or subject to which the compounds of the invention can be administered. In an exemplary embodiment of the present invention, to identify subjects or patients for treatment according to the methods of the invention, accepted screening methods are employed to determine risk factors associated with a targeted or suspected disease or condition or to determine the status of an existing disease or condition in a subject or patient. These screening methods include, for example: conventional work-ups to determine risk factors that may be associated with the targeted or suspected disease or condition. These and other routine methods allow the clinician to select patients in need of therapy using the methods and compounds of the present invention.

As used herein, the expression “biological target” refers to an organ, tissue and/or cell of an organism and/or to the organism itself.

As used herein the term “immunogenic” refers to a substance that is able to elicit an immune response.

The term “substituted” means that an atom or group of atoms formally replaces hydrogen as a “substituent” attached to another group. The term “substituted”, unless otherwise indicated, refers to any level of substitution, e.g.: mono-, di-, tri-, tetra-or penta-substitution, where such substitution is permitted. The substituents are independently selected and substitution may be at any chemically accessible position. It is to be understood that substitution at a given atom is limited by valency. It is to be understood that substitution at a given atom results in a chemically stable molecule. The phrase “optionally substituted” means unsubstituted or substituted. A single divalent substituent, e.g.: oxo, can replace two hydrogen atoms.

The term “alkyl” employed alone or in combination with other terms, refers to a saturated hydrocarbon group that may be straight-chained or branched. Examples of alkyl moieties include, but are not limited to: chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. Examples of alkenyl groups include, but are not limited to: ethenyl, n-propenyl, isopropenyl, n-butenyl, sec-butenyl and the like.

The term “alkoxy”, employed alone or in combination with other terms, refers to a group of formula -O-alkyl, wherein the alkyl group is as defined above. Examples of alkoxy groups include: methoxy, ethoxy, propoxy (e.g.: n-propoxy and isopropoxy), t-butoxy and the like.

All compounds, and pharmaceutically acceptable salts thereof, can be found together with other substances such as water and solvents (e.g.: hydrates and solvates) or can be isolated. When in the solid state, the compounds described herein and salts thereof may occur in various forms and may, for example, take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the invention, or salts thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive: toxicity, irritation, allergic response, or other problem or complication, commensurate with a reasonable benefit/risk ratio.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to: mineral or organic acid salts of basic residues such as amines; alkali or organic salts of acidic residues such as carboxylic acids; and the like. The pharmaceutically acceptable salts of the present invention include the non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like: ether, ethyl acetate, alcohols (e.g.: methanol, ethanol, iso-propanol or butanol) or acetonitrile (MeCN) are preferred. Lists of suitable salts are found in Remington's Pharmaceutical Sciences, 17th Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection and Use, (Wiley, 2002). In some embodiments, the compounds described herein include the N-oxide forms.

All percentages, ratios and proportions herein are by weight, unless otherwise specified. All temperatures are in degrees Celsius (° C) unless otherwise specified.

### Therapeutic Method

The method of the invention preferably comprises administering to a patient a composition to treat a condition in the patient, wherein the composition comprises a metal complex represented by Formula (I) as defined above, including hydrates, solvates, pharmaceutically acceptable salts and prodrugs thereof.

Without wishing to be bound by any theory, it is believed that the composition at least partially reverses or at least partially prevents resistance to a therapy (i.e., an original or primary treatment that is compromised or is susceptible to being compromised by, e.g., MDR) by blocking the specific mechanism of resistance so as to “re-sensitize” the patient to the original treatment. In some embodiments, the composition is useful for the inhibition of efflux pump activity and re-sensitizing targeted cells (e.g., infected cells and/or cancer cells) to drugs and/or other forms of active therapy.

It is further believed that preferred embodiments of the invention induce “Rutherroptosis”, which as defined herein is a process that mimics iron-induced ferroptosis, using metal complexes according to Formula (I), wherein the metal is ruthenium and the ligands include phenanthroline and thiophene. Particularly preferred among these complexes is Ru(4,4′-dimethyl-2,2′-bipyridine)₂(2-(2′,2″:5″,2″-terthiophene)-imidazo[4,5-f][1,10] phenanthroline), which is available from Theralase Technologies of Toronto, Canada, as a dichloride salt with (RUTHERRIN) or without transferrin (RUVIDAR).

Inert Ru(II) polypyridyl complexes hold tremendous potential as chemotherapeutic agents against cancer and infectious diseases, both in the presence of light and/or radiation—i.e., as sensitizers for PDT and RDT—and in their absence.

Hence, in certain embodiments, the present invention provides a method of treating a drug resistant tumor and/or recurrent tumors and/or infectious diseases comprising administering a metal complex of Formula (I) to a mammalian patient suffering from these disorders under conditions effective to treat the drug resistant tumor and/or persistent tumors and/or infectious diseases via induction of a particular intracellular process, which is (to at least some extent) mimicking the ferroptosis pathway.

In normal cells, ROS levels are kept low due to the antioxidant systems that maintain redox balance due to oxidative phosphorylation metabolic pathway (Schumacker. Reactive oxygen species in cancer: a dance with the devil. Cancer Cell. 2015; 27(2):156-157. doi: 10.1016/j.ccell.2015.01.007). In cancer cells, due to aerobic glycolysis, Warburg effect, the level of ROS increases to meet the need of malignant proliferation and progression, but may still stay below the threshold to avoid cytotoxicity (Marengo et al. Redox homeostasis and cellular antioxidant systems: crucial players in cancer growth and therapy. Oxidative Medicine and Cellular Longevity. 2016; 2016:16. doi: 10.1155/2016/6235641.6235641).

RUVIDAR and RUTHERRIN have been shown to exhibit a lower systemic toxicity and linked with a higher selectivity towards, particularly stem and/or high grade, hypoxic cancer cells, which is knowingly expressing the highest level of transferrin receptor. See Gao et al. The Mechanisms of Ferroptosis Under Hypoxia. Cell Mol Neurobiol 43, 3329-3341 (2023). https://doi.org/10.1007/s10571-023-01388-8.

Without activation by light and/or radiation, the activation for these highly versatile metal-ligands complexes in cancer cells can be driven by high redox/oxidative potential, which could carry out their significant cytotoxic activity in cancer cells through interactions with the cellular redox/oxidative Warburg's homeostasis.

Therefore, in yet another aspect, the present invention provides a method of treating a drug resistant tumor and/or recurrent tumors and/or infective diseases comprising administering a compound of Formula (I) to a patient suffering from these disorders under conditions effective to treat the drug resistant tumor and/or persistent tumors and/or infectious diseases via induction of redox/oxidative intracellular process under hypoxia and/or anaerobic conditions.

Radiotherapy uses radiation to irradiate tumor tissues and kill tumor cells. On the one hand, radiation acts directly on cells and instantly produces a large number of free radicals. On the other hand, it indirectly produces lasting and severe therapeutic effects through the redox reaction of water (Spitz et al. Metabolic oxidation/reduction reactions and cellular responses to ionizing radiation: a unifying concept in stress response biology. Cancer and Metastasis Reviews. 2004; 23(3/4):311-322. doi: 10.1023/B: CANC.0000031769.14728.bc).

Due to the high content of water in cells, when water absorbs the energy of low-LET rays, a redox reaction occurs, and a large number of free radicals and free electrons are produced. The free radicals and electrons generated initiate cascade reactions that produce OH, H₂O₂, and O_{2·}³¹ , significantly increasing the level of ROS(Bielski et al. Highlights of current research involving superoxide and perhydroxyl radicals in aqueous solutions. International Journal of Radiation Biology. 2009; 59(2):291-319. doi: 10.1080/09553009114550301). Notably, oxidative changes can persist for several months after initial radiotherapy. This feature is related to the continuous generation of ROS and its heritability in the offspring of irradiated cells and obviously enhances the curative effect (Tamminga et al. Role of DNA damage and epigenetic DNA methylation changes in radiation-induced genomic instability and bystander effects in germline in vivo. Current Molecular Pharmacology. 2011; 4(2):115-125. doi: 10.2174/1874467211104020115).

The basic idea behind activation of metal complexes, such as RUVIDAR and RUTHERRIN, is based on the induction of photoconversion of the metal complexes that triggers the production of ROS and free radicals, leading to the killing of unwanted cells. The main difficulties related to use of light-based PDT for clinical applications are due to the intrinsic process, such as absorption and scattering of light in penetrating tissues, the low concentration of FDA-approved PDCs, particularly pro-drugs like ALA, which must be effectively converted to (under the Warburg's metabolic environment with the lack of oxygen) to the active pharmaceutical ingredient PPIX.

Upon the higher charge particle energy about >633 KeV emitted by different radiation sources the Vavilov-Cherenkov effect (photon emission), which is possible to enhance the radiation effect on activation of PDCs, such as RUVIDAR and RUTHERRIN, is observed. A classic example of Cherenkov radiation is the characteristic blue glow of an underwater nuclear reactor. Its cause is similar to the cause of a sonic boom, the sharp sound heard when faster-than-sound and/or light movement occurs (Cherenkov. (1934). “Visible emission of clean liquids by action of γ radiation”. Doklady Akademii Nauk SSSR. 2: 451. Reprinted in Selected Papers of Soviet Physicists, Usp. Fiz. Nauk 93 (1967) 385. V sbornike: Pavel Alekseyevich Čerenkov: Chelovek i Otkrytie pod redaktsiej A. N. Gorbunova i E. P. Čerenkovoj, M., Nauka, 1999, s. 149-153. (ref Archived Oct. 22, 2007, at the Wayback Machine)).

The mainstream treatment for cancer, chemotherapy, also often works by changing the redox state of cancer cells. Quite a few chemotherapeutics induce oxidative stress and ROS-mediated cell damage in cancer cells by increasing ROS above the threshold to yield an anticancer effect [Pelicano et al. ROS stress in cancer cells and therapeutic implications. Drug Resistance Updates. 2004; 7(2):97-110. doi: 10.1016/j.drup.2004.01.004). Most of these drugs produce ROS directly in cancer cells to increase the level of ROS. The first drug developed to achieve therapeutic effects by producing ROS was procarbazine. Procarbazine can be oxidised in aqueous solution and produce H₂O₂ and ·OH.

When coordinating with ionizing radiation, procarbazine forms unstable peroxides to damage DNA in vitro (Berneis et al. The enhancement of the after effect of ionizing radiation by a cytotoxic methylhydrazine derivative. European Journal of Cancer. 2004; 40(13): 928-1933. doi: 10.1016/j.cjca.2004.04.013). It was approved for the treatment of primary brain tumours and other diseases 60 years ago (Behrend. Patients with primary brain tumors. Oncology Nursing Forum. 2014; 4(3): 335-336. doi: 10.1188/14.Onf.335-336).

Nowadays, drugs like anthracycline (Songbo et al. Oxidative stress injury in doxorubicin-induced cardiotoxicity. Toxicology Letters. 2019; 307:41-48. doi: 10.1016/j.toxlet.2019.02.013) are widely used in cancer treatment to promote ROS production.

Therefore, in yet another aspect, the present invention provides a method of treating a drug resistant tumor and/or recurrent tumors and/or infective diseases comprising administering a compound of Formula (I) to a mammalian patient suffering from these disorders under conditions effective to treat the drug resistant tumor and/or persistent tumors and/or infectious diseases via induction of redox/oxidative intracellular process under hypoxia and/or anaerobic conditions. In some embodiments, said administering is carried out in combination with another cancer or anti-infective therapy.

Tumor cells develop various mechanisms to resist immune attack. The immune pressure exerted against the tumor during immunotherapy treatments is strongly suggested by the escape mechanisms developed by the tumor, especially against the attack of cytotoxic T lymphocytes (De Guillebon et al. (2020), Beyond the concept of cold and hot tumors for the development of novel predictive biomarkers and the rational design of immunotherapy combination. Int. J. Cancer, 147:1509-1518. https://doi.org/10.1002/ijc.32889).

This immune resistance can be reversed and/or uniquely modulated by the dual checkpoint inhibition (e.g., CD47 and PD-L1 inhibition) with administering a compound of formula (I) to a mammalian patient. This inventive therapeutic approach will also improve patient outcomes over current PD-L1 and CD47-targeted therapies.

Hence, in some embodiments, the composition of the invention is useful for the inhibition of several immunologic checkpoints and re-sensitizing the cancer cells and/or cancer-resistant cells to drugs and/or other forms of active therapy and/or immunotherapy. In preferred embodiments, said administering is carried out in combination with another cancer or anti-infective therapy.

In certain embodiments, the at least one API is at least one member selected from the group consisting of Abecma (Idecabtagene Vicleucel), Abemaciclib, Abiraterone Acetate, Abraxane (Paclitaxel Albumin-stabilized Nanoparticle Formulation), ABVD, ABVE, ABVE-PC, AC, Acalabrutinib, AC-T, Actemra (Tocilizumab), Adagrasib, Adcetris (Brentuximab Vedotin), ADE, Ado-Trastuzumab Emtansine, Adriamycin (Doxorubicin Hydrochloride), Adstiladrin (Nadofaragene Firadenovec-vncg), Afatinib Dimaleate, Afinitor (Everolimus), Akcega (Niraparib Tosylate Monohydrate and Abiraterone Acetate), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Alkeran for Injection (Melphalan Hydrochloride), Alkeran Tablets (Melphalan), Aloxi (Palonosetron Hydrochloride), Alpelisib, Alunbrig (Brigatinib), Alymsys (Bevacizumab), Ameluz (Aminolevulinic Acid Hydrochloride), Amifostinc, Aminolevulinic Acid Hydrochloride, Amivantamab-vmjw, Amtagvi (Lifileucel), Anastrozole, Apalutamide, Aprepitant, Aranesp (Darbepoetin Alfa), Aredia (Pamidronate Disodium), Arimidex (Anastrozole), Aromasin (Exemestane), Arranon (Nelarabine), Arsenic Trioxide, Arzerra (Ofatumumab), Asciminib Hydrochloride, Asparaginase Erwinia Chrysanthemi, Asparaginase Erwinia Chrysanthemi (Recombinant)-rywn, Asparlas (Calaspargase Pegol-mknl), Atezolizumab, Augtyro (Repotrectinib), Avapritinib, Avastin (Bevacizumab), Avelumab, Axicabtagene Ciloleucel, Axitinib, Ayvakit (Avapritinib), Azacitidine, Azedra (Iobenguane I 131), Balversa (Erdafitinib), Bavencio (Avelumab), BEACOPP, Belcodaq (Belinostat), Belinostat, Belzutifan, Bendamustine Hydrochloride, Bendeka (Bendamustine Hydrochloride), BEP, Besponsa (Inotuzumab Ozogamicin), Besremi (Ropeginterferon Alfa-2b-njft), Bevacizumab, Bexarotene, Bicalutamide, BiCNU (Carmustinc), Binimctinib, Bleomycin Sulfate, Blinatumomab, Blincyto (Blinatumomab), Bortezomib, Bosulif (Bosutinib), Bosutinib, Braftovi (Encorafenib), Brentuximab Vedotin, Brexucabtagene Autoleucel, Breyanzi (Lisocabtagene Maralcucel), Brigatinib, Brukinsa (Zanubrutinib), BuMel, Busulfan, Busulfex (Busulfan), Cabazitaxel, Cablivi (Caplacizumab-yhdp), Cabometyx (Cabozantinib-S-Malate), Cabozantinib-S-Malate, CAF, Calaspargase Pegol-mknl, Calquence (Acalabrutinib), Campath (Alemtuzumab), Camptosar (Irinotecan Hydrochloride), Capecitabine, Capivasertib, Caplacizumab-yhdp, Capmatinib Hydrochloride, CAPOX, Carac (Fluorouracil Topical), Carboplatin, CARBOPLATIN-TAXOL, Carfilzomib, Carmustine, Carmustine Implant, Carvykti (Ciltacabtagene Autoleucel), Casodex (Bicalutamide), CEM, Cemiplimab-rwlc, Ceritinib, Cervarix (Recombinant HPV Bivalent Vaccine), Cetuximab, CEV, Chlorambucil, CHLORAMBUCIL-PREDNISONE, CHOP, Ciltacabtagene Autoleucel, Cisplatin, Cladribine, Clofarabine, Clolar (Clofarabine), CMF, Cobimctinib Fumarate, Columvi (Glofitamab-gxbm), Cometriq (Cabozantinib-S-Malate), COPDAC, Copiktra (Duvelisib), COPP, COPP-ABV, Cosmegen (Dactinomycin), Cotellic (Cobimetinib Fumarate), Crizotinib, CVP, Cyclophosphamide, Cyramza (Ramucirumab), Cytarabine, Dabrafenib Mesylate, Dacarbazine, Dacogen (Decitabine), Dacomitinib, Dactinomycin, Danyelza (Naxitamab-gqgk), Daratumumab, Daratumumab and Hyaluronidase-fihj, Darbepoetin Alfa, Darolutamide, Darzalex (Daratumumab), Darzalex Faspro (Daratumumab and Hyaluronidase-fihj), Dasatinib, Daunorubicin Hydrochloride, Daunorubicin Hydrochloride and Cytarabine Liposome, Daurismo (Glasdegib Malcate), Decitabine, Decitabine and Cedazuridine, Defibrotide Sodium, Defitelio (Defibrotide Sodium), Degarelix, Denileukin Diftitox, Denosumab, Dexamethasone, Dexrazoxane Hydrochloride, Dinutuximab, Docetaxel, Dostarlimab-gxly, Doxil (Doxorubicin Hydrochloride Liposome), Doxorubicin Hydrochloride, Doxorubicin Hydrochloride Liposome, Durvalumab, Duvelisib, Eflornithinc Hydrochloride, Efudex (Fluorouracil Topical), Elacestrant Dihydrochloride, Elahere (Mirvetuximab Soravtansine-gynx), Eligard (Leuprolide Acetate), Elitek (Rasburicase), Ellence (Epirubicin Hydrochloride), Elotuzumab, Eloxatin (Oxaliplatin), Elranatamab-bcmm, Elrexfio (Elranatamab-bcmm), Eltrombopag Olamine, Elzonris (Tagraxofusp-erzs), Emapalumab-Izsg, Emend (Aprepitant), Empliciti (Elotuzumab), Enasidenib Mesylate, Encorafenib, Enfortumab Vedotin-ejfv, Enhertu (Fam-Trastuzumab Deruxtecan-nxki), Entrectinib, Enzalutamide, Epcoritamab-bysp, Epirubicin Hydrochloride, Epkinly (Epcoritamab-bysp), EPOCH, Epoctin Alfa, Epogen (Epoctin Alfa), Erbitux (Cetuximab), Erdafitinib, Eribulin Mesylate, Erivedge (Vismodegib), Erleada (Apalutamide), Erlotinib Hydrochloride, Erwinaze (Asparaginase Erwinia chrysanthemi), Ethyol (Amifostine), Etopophos(Etoposide Phosphate), Etoposide, Etoposide Phosphate, Everolimus, Evista (Raloxifene Hydrochloride), Evomcla (Melphalan Hydrochloride), Exemestane, 5-FU (Fluorouracil Injection), 5-FU (Fluorouracil Topical), Fam-Trastuzumab Deruxtecan-nxki, Fareston (Toremifene), Faslodex (Fulvestrant), FEC, Fedratinib Hydrochloride, Femara (Letrozole), Filgrastim, Firmagon (Degarelix), Fludarabine Phosphate, Fluoroplex (Fluorouracil Topical), Fluorouracil Injection, Fluorouracil Topical, Flutamide, FOLFIRI, FOLFIRI-BEVACIZUMAB, FOLFIRI-CETUXIMAB, FOLFIRINOX, FOLFOX, Folotyn (Pralatrexate), Fostamatinib Disodium, Fotivda (Tivozanib Hydrochloride), Fruquintinib, Fruzaqla (Fruquintinib), Fulphila (Pegfilgrastim), FU-LV, Fulvestrant, Futibatinib, Fyarro (Sirolimus Protein-Bound Particles), Gamifant (Emapalumab-lzsg), Gardasil (Recombinant HPV Quadrivalent Vaccine), Gardasil 9 (Recombinant HPV Nonavalent Vaccine), Gavreto (Pralsetinib), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gilteritinib Fumarate, Glasdegib Maleate, Gleevec (Imatinib Mesylate), Gliadel Wafer (Carmustine Implant), Glofitamab-gxbm, Glucarpidase, Goserelin Acetate, Granisetron, Granisetron Hydrochloride, Granix (Filgrastim), Halaven (Eribulin Mesylate), Hemangeol (Propranolol Hydrochloride), Hepzato (Melphalan Hydrochloride), Herceptin Hylecta (Trastuzumab and Hyaluronidase-oysk), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurca), Hydroxyurea, Hyper-CVAD, Ibrance (Palbociclib), Ibritumomab Tiuxctan, Ibrutinib, ICE, Iclusig (Ponatinib Hydrochloride), Idamycin PFS (Idarubicin Hydrochloride), Idarubicin Hydrochloride, Idecabtagene Vicleucel, Idelalisib, Idhifa (Enasidenib Mesylate), Ifex (Ifosfamide), Ifosfamide, IL-2 (Aldesleukin), Imatinib Mesylate, Imbruvica (Ibrutinib), Imfinzi (Durvalumab), Imiquimod, Imjudo (Tremelimumab-actl), Imlygic (Talimogene Laherparepvec), Infugem (Gemcitabine Hydrochloride), Inlyta (Axitinib), Inotuzumab Ozogamicin, Inqovi (Decitabine and Cedazuridine), Inrebic (Fedratinib Hydrochloride), Interferon Alfa-2b, Recombinant, Interleukin-2 (Aldesleukin), Intron A (Recombinant Interferon Alfa-2b), Iobenguane I 131, Ipilimumab, Iressa (Gefitinib), Irinotecan Hydrochloride, Irinotecan Sucrosofate, Isatuximab-irfc, Istodax (Romidepsin), Ivosidenib, Iwilfin (Eflornithine Hydrochloride), Ixabepilone, Ixazomib Citrate, Ixempra (Ixabepilone), Jakafi (Ruxolitinib Phosphate), Jaypirca (Pirtobrutinib), JEB, Jelmyto (Mitomycin), Jemperli (Dostarlimab-gxly), Jevtana (Cabazitaxel), Kadcyla (Ado-Trastuzumab Emtansine), Kopivance (Palifermin), Keytruda (Pembrolizumab), Kimmtrak (Tebentafusp-tebn), Kisqali (Ribociclib), Koselugo (Sclumetinib Sulfate), Krazati (Adagrasib), Kymriah (Tisagenlecleucel), Kyprolis (Carfilzomib), Lanreotide Acetate, Lapatinib Ditosylate, Larotrectinib Sulfate, Lenalidomide, Lenvatinib Mesylate, Lenvima (Lenvatinib Mesylate), Letrozole, Leucovorin Calcium, Leukeran (Chlorambucil), Leuprolide Acetate, Levulan Kerastick (Aminolevulinic Acid Hydrochloride), Libtayo (Cemiplimab-rwlc), Lifileucel, Lisocabtagene Maraleucel, Lomustine, Loncastuximab Tesirine-lpyl, Lonsurf (Trifluridine and Tipiracil Hydrochloride), Loqtorzi (Toripalimab-tpzi), Lorbrena (Lorlatinib), Lorlatinib, Lumakras (Sotorasib), Lumoxiti (Moxctumomab Pasudotox-tdfk), Lunsumio (Mosunetuzumab-axgb), Lupron Depot (Leuprolide Acetate), Lurbinectedin, Luspatercept-aamt, Lutathera (Lutetium Lu 177-Dotatate), Lutetium (Lu 177-Dotatate), Lutetium Lu 177 Vipivotide Tetraxctan, Lynparza (Olaparib), Lytgobi (Futibatinib), Margenza (Margetuximab-cmkb), Margetuximab-cmkb, Matulane (Procarbazine Hydrochloride), Mechlorethamine Hydrochloride, Megestrol Acetate, Mekinist (Tramctinib Dimethyl Sulfoxide), Mektovi (Binimetinib), Melphalan, Melphalan Hydrochloride, Mercaptopurine, Mesna, Mesnex (Mesna), Methotrexate Sodium, Methylnaltrexone Bromide, Midostaurin, Mirvetuximab Soravtansine-gynx, Mitomycin Mitoxantrone Hydrochloride, Mogamulizumab-kpkc, Momelotinib Dihydrochloride Monohydrate, Monjuvi (Tafasitamab-cxix), MOPP, Mosunetuzumab-axgb, Moxctumomab Pasudotox-tdfk, Mozobil (Plerixafor), MVAC, Mvasi (Bevacizumab), Myleran (Busulfan), Mylotarg (Gemtuzumab Ozogamicin), Nadofaragene Firadenovec-vncg, Nanoparticle Paclitaxel (Paclitaxel Albumin-stabilized Nanoparticle Formulation), Naxitamab-gqgk, Necitumumab, Nelarabine, Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Niraparib Tosylate Monohydrate and Abiraterone Acetate, Nirogacestat Hydrobromide, Nivestym (Filgrastim), Nivolumab, Nivolumab and Relatlimab-rmbw, Nplate (Romiplostim), Nubeqa (Darolutamide), Nyvepria (Pegfilgrastim), Obinutuzumab, Odomzo (Sonidegib), OEPA, Ofatumumab, OFF, Ogsiveo (Nirogacestat Hydrobromide), Ojjaara (Momelotinib Dihydrochloride Monohydrate), Olaparib, Olutasidenib, Omacetaxinc Mepesuccinate, Oncaspar (Pegaspargase), Ondansetron Hydrochloride, Onivyde (Irinotecan Sucrosofate), Ontak (Denilcukin Diftitox), Onureg (Azacitidine), Opdivo (Nivolumab), Opdualag (Nivolumab and Relatlimab-rmbw), OPPA, Orgovyx (Relugolix), Orserdu (Elacestrant Dihydrochloride), Osimertinib Mesylate, Oxaliplatin, Paclitaxel, Paclitaxel Albumin-stabilized Nanoparticle Formulation, Pacritinib Citrate, PAD, Padcev (Enfortumab Vedotin-cjfv), Palbociclib, Palifermin, Palonosetron Hydrochloride, Palonosetron Hydrochloride and Netupitant, Pamidronate Disodium, Panitumumab, Paraplatin (Carboplatin), Pazopanib Hydrochloride, PCV, PEB, Pegaspargase, Pegfilgrastim, Pemazyre (Pemigatinib), Pembrolizumab, Pemetrexed Disodium, Pemigatinib, Perjeta (Pertuzumab), Pertuzumab, Pertuzumab, Trastuzumab, and Hyaluronidase-zzxf, Pexidartinib Hydrochloride, Phesgo (Pertuzumab, Trastuzumab, and Hyaluronidase-zzxf), Piqray (Alpelisib), Pirtobrutinib, Plerixafor, Pluvicto (Lutetium Lu 177 Vipivotide Tetraxetan), Polatuzumab Vedotin-piiq, Polivy (Polatuzumab Vedotin-piiq), Pomalidomide, Pomalyst (Pomalidomide), Ponatinib Hydrochloride, Portrazza (Necitumumab), Poteligco (Mogamulizumab-kpkc), Pralatrexate, Pralsetinib, Prednisone, Procarbazine Hydrochloride, Procrit (Epoctin Alfa), Proleukin (Aldesleukin), Prolia (Denosumab), Promacta (Eltrombopag Olamine), Propranolol Hydrochloride, Provenge (Sipuleucel-T), Purincthol (Mercaptopurine), Purixan (Mercaptopurine), Qinlock (Ripretinib), Quizartinib Dihydrochloride, Radium 223 Dichloride, Raloxifene Hydrochloride, Ramucirumab, Rasburicase, Ravulizumab-cwvz, Reblozyl (Luspatercept-aamt), R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), Relugolix, R-EPOCH, Repotrectinib, Retacrit (Epoetin Alfa), Retevmo (Selpercatinib), Retifanlimab-dlwr, Revlimid (Lenalidomide), Rezlidhia (Olutasidenib), Riabni (Rituximab), Ribociclib, R-ICE, Ripretinib, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Ropeginterferon Alfa-2b-njft, Rozlytrek (Entrectinib), Rubraca (Rucaparib Camsylate), Rucaparib Camsylate, Ruxience (Rituximab), Ruxolitinib Phosphate, Rybrevant (Amivantamab-vmjw), Rydapt (Midostaurin), Rylaze (Asparaginase Erwinia Chrysanthemi [Recombinant]-rywn), Sacituzumab Govitecan-hziy, Sancuso (Granisetron), Sarclisa (Isatuximab-irfc), Sclerosol Intrapleural Aerosol (Talc), Selinexor, Selpercatinib, Selumetinib Sulfate, Scemblix (Asciminib Hydrochloride), Siltuximab, Sipulcucel-T, Sirolimus Protein-Bound Particles, Soltamox (Tamoxifen Citrate), Somatuline Depot (Lanreotide Acetate), Sonidegib, Sorafenib Tosylate, Sotorasib, Sprycel (Dasatinib), STANFORD V, Sterile Talc Powder (Talc), Steritalc (Talc), Stivarga (Regorafenib), Sunitinib Malate, Sustol (Granisctron), Sutent (Sunitinib Malate), Sylvant (Siltuximab), Synribo (Omacetaxine Mepesuccinate), Tabloid (Thioguanine), Tabrecta (Capmatinib Hydrochloride), TAC, Tafasitamab-cxix, Tafinlar (Dabrafenib Mesylate), Tagraxofusp-erzs, Tagrisso (Osimertinib Mesylate), Talazoparib Tosylate, Talc, Talimogene Laherparepvec, Talquetamab-tgvs, Talvey (Talquetamab-tgvs), Talzenna (Talazoparib Tosylate), Tamoxifen Citrate, Tarceva (Erlotinib Hydrochloride), Targretin (Bexarotene), Tasigna (Nilotinib), Tavalisse (Fostamatinib Disodium), Taxotere (Docetaxel), Tazemetostat Hydrobromide, Tazverik (Tazemetostat Hydrobromide), Tebentafusp-tebn, Tecartus (Brexucabtagene Autoleucel), Tecentriq (Atezolizumab), Teclistamab-cqyv, Tecvayli (Teclistamab-cqyv), Temodar (Temozolomide), Temozolomide, Temsirolimus, Tepadina (Thiotepa), Tepmetko (Tepotinib Hydrochloride), Tepotinib Hydrochloride, Tevimbra (Tislelizumab-jsgr), Thalidomide, Thalomid (Thalidomide), Thioguanine, Thiotepa, Tibsovo (Ivosidenib), Tisagenlecleucel, Tislelizumab-jsgr, Tisotumab Vedotin-tftv, Tivdak (Tisotumab Vedotin-tftv), Tivozanib Hydrochloride, Tocilizumab, Tolak (Fluorouracil Topical), Topotecan Hydrochloride, Toremifene, Toripalimab-tpzi, Torisel (Temsirolimus), Totect (Dexrazoxane Hydrochloride), TPF, Trabectedin, Tramctinib Dimethyl Sulfoxide, Trastuzumab, Trastuzumab and Hyaluronidase-oysk, Treanda (Bendamustine Hydrochloride), Tremelimumab-actl, Trexall (Methotrexate Sodium), Trifluridine and Tipiracil Hydrochloride, Trisenox (Arsenic Trioxide), Trodelvy (Sacituzumab Govitecan-hziy), Truqap (Capivasertib), Truxima (Rituximab), Tucatinib, Tukysa (Tucatinib), Turalio (Pexidartinib Hydrochloride), Tykerb (Lapatinib Ditosylate), Ultomiris (Ravulizumab-cwvz), Undencyca (Pegfilgrastim), Unituxin (Dinutuximab), Uridine Triacetate, VAC, Valrubicin, Valstar (Valrubicin), VAMP, Vandetanib, Vanflyta (Quizartinib Dihydrochloride), Varubi (Rolapitant Hydrochloride), Vectibix (Panitumumab), VeIP, Velcade (Bortezomib), Vemurafenib, Venclexta (Venetoclax), Venetoclax, Verzenio (Abemaciclib), Vidaza (Azacitidine), Vinblastine Sulfate, Vincristine Sulfate, Vinorelbine Tartrate, VIP, Vismodegib, Vistogard (Uridine Triacetate), Vitrakvi (Larotrectinib Sulfate), Vizimpro (Dacomitinib), Vonjo (Pacritinib Citrate), Voraxaze (Glucarpidase), Vorinostat, Votrient (Pazopanib Hydrochloride), Vyxcos (Daunorubicin Hydrochloride and Cytarabine Liposome), Welireg (Belzutifan), Xalkori (Crizotinib), Xatmep (Methotrexate Sodium), Xeloda (Capecitabine), XELIRI, XELOX, Xgeva (Denosumab), Xofigo (Radium 223 Dichloride), Xospata (Gilteritinib Fumarate), Xpovio (Selinexor), Xtandi (Enzalutamide), Yervoy (Ipilimumab), Yescarta (Axicabtagene Ciloleucel), Yondelis (Trabectedin), Yonsa (Abiraterone Acetate), Zaltrap (Ziv-Aflibercept), Zanubrutinib, Zarxio (Filgrastim), Zejula (Niraparib Tosylate Monohydrate), Zelboraf (Vemurafenib), Zepzelca (Lurbinectedin), Zevalin (Ibritumomab Tiuxetan), Ziextenzo (Pegfilgrastim), Zinecard (Dexrazoxane Hydrochloride), Zirabev (Bevcizumab), Ziv-Aflibercept, Zofran (Ondansetron Hydrochloride), Zoladex (Goserelin Acetate), Zoledronic Acid, Zolinza (Vorinostat), Zometa (Zoledronic Acid), Zyclara (Imiquimod), Zydelig (Idelalisib), Zykadia (Ceritinib), Zynlonta (Loncastuximab Tesirine-lpyl), Zynyz (Retifanlimab-dlwr) and Zytiga (Abiraterone Acetate).

In certain embodiments, the at least one API is selected from the group consisting of a checkpoint inhibitor, such as, e.g., PD-1, PD-L1, PD-L2, CTLA4, TIM3, CEACAM (i.e.: CEACAM-1, CEACAM-3 and/or CEACAM-5), LAG3, VISTA, BTLA, TIGIT, LAIRI, CD 160, 2B4, CD80, CD86, B7-H3 (CD276), B7-H4 (VTCNI), HVEM (i.e.: TNFRSF14 or CD270), KIR, A2aR, MHC class I, MHC class II, GAL9, adenosine, TGF (i.e.: TGF beta or an anti-PD-1 antibody or fragment thereof, (i.e.: Pembrolizumab).

In certain embodiments, the at least one API is selected from the group consisting of a non-replicating recombinant viral gene therapy, an attenuated recombinant gene therapy and/or gene therapy; including: CAR-T cells, viral vectors and/or nonviral vectors, further characterized as polyplex, lipoplex, lipid-polymer hybrid (lipopolyplex) and organic-inorganic hybrid vectors. Nonviral vectors include various biodegradable and/or biocompatible formulations. Inorganic nanocarriers include formulations that are chemically and thermally stable, easily controllable in particle size, shape and/or structure. Non-viral and viral systems/vectors include positive charged formulations (i.e.: metals, cationic liposomes, polymers, etc.) allowing them the ability to interact and bind to negatively charged lipids and/or DNA.

In certain embodiments, the at least one API is a carbohydrate, mRNA, antibody, nucleic acid, protein, biologic (synthetic, non-synthetic, non-biologic component or device and/or their combination), modified cells and/or unmodified cells (including microorganisms) and/or their recombinants.

In certain embodiments, the at least one API is a chemotherapeutic agent (e.g., Bacillus Calmette-Guérin (BCG)), which is systemically and/or intratumorally administered.

In certain embodiments, the at least one API is an antibiotic selected from the group consisting of Aminoglycosides, Carbapenems, Cephalosporins, Fluoroquinolones, Glycopeptides, Lipoglycopeptides, Macrolides, Monobactams, Oxazolidinones, Penicillins, Polypeptides, Rifamycins, Sulfonamides, Streptogramins and Tetracyclines.

### Composition Comprising the Metal Complex

The composition comprises a pharmaceutically acceptable excipient in addition to the metal complex. For the purposes of the present invention the terms “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable and functional pharmaceutical, serving not only as part of the overall vehicle for delivery, but also as a means for achieving effective absorption by the recipient of the active ingredient. An excipient may fill a role as simple and direct as being an inert filler, or an excipient as used herein may, for example, be part of a pH stabilizing system or coating to insure delivery of the ingredients safely to the tissue.

Examples of such excipients or carriers are well known to those skilled in the art and can be prepared in accordance with acceptable pharmaceutical procedures, such as, for example, those described in Remington's Pharmaceutical Sciences, 17^{th} edition, ed. Alfonoso R. Gennaro, Mack Publishing Company, Easton, PA (1985), the entire disclosure of which is incorporated by reference herein for all purposes. Supplementary active ingredients can also be incorporated into the pharmaceutical compositions.

Active agents used in the invention (e.g., the metal complex and at least one active pharmaceutical ingredient whose efficacy is to be preserved or revived) can be administered: orally, intravenously, intravesically, lingually, intratumorally, topically or parenterally, neat or in combination with conventional pharmaceutical carriers. Applicable solid carriers can include one or more substances which can also act as: flavoring agents, lubricants, solubilizers, suspending agents, fillers, glidants, compression aids, binders or tablet-disintegrating agents or encapsulating materials. The active agents can be formulated in a conventional manner, for example, in a manner similar to that used for known active agents. Oral formulations containing an active agent disclosed herein can comprise any conventionally used oral form, including: tablets, capsules, buccal forms, troches, lozenges and oral liquids, suspensions or solutions. In powders, the carrier can be a finely divided solid, which is an admixture with a finely divided active agent. In tablets, an active agent disclosed herein can be mixed with a carrier having the necessary compression properties in suitable proportions and compacted in the shape and size desired. The powders and tablets can contain up to 99% of the active agent.

Capsules can contain mixtures of one or more compound(s) and/or compositions disclosed herein with inert filler(s) and/or diluent(s) such as: pharmaceutically acceptable starches (i.e.: corn, potato or tapioca starch), sugars, artificial sweetening agents, powdered celluloses (i.e.: crystalline and microcrystalline celluloses), flours, gelatins, gums and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to: magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes and ion exchange resins. Surface modifying agents include nonionic and anionic surface modifying agents. Representative examples of surface modifying agents include, but are not limited to: poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate and triethanolamine. Oral formulations herein can utilize standard delay or time-release formulations to alter the absorption of the compound(s) and/or compositions. The oral formulation can also consist of administering an active agent disclosed herein in water or fruit juice, containing appropriate solubilizers or emulsifiers, as needed.

Liquid carriers can be used in preparing solutions, suspensions, emulsions, syrups, elixirs and for inhaled delivery. An active agent of the invention can be dissolved or suspended in a pharmaceutically acceptable liquid carrier such as: water, an organic solvent, a mixture of both or a pharmaceutically acceptable oil or fat. The liquid carrier can contain other suitable pharmaceutical additives such as: solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to: water (particularly containing additives as described herein, for example, cellulose derivatives such as a sodium carboxymethyl cellulose solution), alcohols (including monohydric alcohols and polyhydric alcohols, for example, glycols) and their derivatives, and oils (i.e.: fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example: intramuscular, intraperitoneal, topical or subcutaneous injection. Sterile solutions can also be administered intravenously. Compositions for oral administration can be in either liquid or solid form.

In certain embodiments, the pharmaceutical composition is in unit dosage form, for example as: tablets, capsules, powders, solutions, suspensions, emulsions, granules or suppositories. In such form, the pharmaceutical composition can be sub-divided into unit dose(s) containing appropriate quantities of the active agent. The unit dosage forms can be packaged compositions, for example: packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of each active agent to about 500 mg/kg of each active agent and can be given in a single dose or in two or more doses. Such doses can be administered in any manner useful in directing the compound(s) and/or composition(s) to the target tissue and/or bloodstream of the patient, including: orally, via implants, parenterally (including intravenous, intraperitoneal, topical and subcutaneous injections), rectally, vaginally and transdermally.

When administered for the treatment or inhibition of a particular disease state or disorder, it is understood that an effective dosage can vary depending upon the particular active agent utilized, the mode of administration and severity of the condition being treated, as well as the various physical factors related to the individual being treated. In therapeutic applications, an active agent can be provided to a patient already suffering from a disease in an amount sufficient to heal or at least partially ameliorate the symptoms of the disease and its complications. The dosage to be used in the treatment of a specific patient typically must be subjectively determined by the attending physician. The variables involved include the specific condition and its state as well as the: physical size, age, gender, health status and response pattern of the patient.

In some cases, it may be desirable to administer the active agents directly to the airways of the patient, using devices such as, but not limited to: metered dose inhalers, breath-operated inhalers, multidose dry-powder inhalers, pumps, squeeze-actuated nebulized spray dispensers, aerosol dispensers and aerosol nebulizers. For administration by intranasal or intrabronchial inhalation, the active agent(s) can be formulated into a liquid composition, a solid composition, or an aerosol composition. The liquid composition can include, by way of illustration, one or more active agents dissolved, partially dissolved or suspended in one or more pharmaceutically acceptable solvents and can be administered by, for example, a pump or a squeeze-actuated nebulized spray dispenser. The solvents can be administered by, for example, isotonic saline or bacteriostatic water. The solid composition can be administered, by way of illustration, a powder preparation including one or more active agents intermixed with lactose or other inert powders that are acceptable for intrabronchial use, and can be administered by, for example, an aerosol dispenser or a device that breaks or punctures a capsule encasing the solid active agent and delivers the solid active agent for inhalation. The aerosol active agent can include, by way of illustration, one or more active agents, propellants, surfactants and co-solvents, and can be administered by, for example, a metered device. The propellants can be a chlorofluorocarbon, a hydrofluoroalkane, or other propellants that are physiologically and environmentally acceptable.

The active agents of the invention can be administered parenterally or intraperitoneally. Solutions or suspensions of these active agents or pharmaceutically acceptable salts, hydrates, or esters thereof can be prepared in water suitably mixed with a surfactant such as hydroxylpropylcellulose. Dispersions can also be prepared in propylene glycol, glycerol, liquid polyethylene glycols and/or mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In certain embodiments, the form can be sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example: water, ethanol, polyol (i.e.: propylene glycol, glycerol and liquid polyethylene glycol) and/or suitable mixtures thereof in oils.

Active agents described herein can be administered transdermally (i.e.: administered across the surface of the body and the inner linings of bodily passages including epithelial and mucosal tissues). Such administration can be carried out using the active agents of the invention including pharmaceutically acceptable: salts, hydrates, or esters thereof, in lotions, creams, foams, patches, suspensions, solutions and/or suppositories (rectal and vaginal).

Transdermal administration can be accomplished through the use of a transdermal patch containing an active agent disclosed herein, and a carrier that can be inert to the active agent, can be non-toxic to the skin and can allow delivery of the active agent for systemic absorption into the blood stream via the skin. The carrier can take any number of forms such as creams and ointments, pastes, gels and occlusive devices. The creams and ointments can be viscous liquid or semisolid emulsions of either the oil-in-water or water-in-oil type. Pastes comprised of absorptive powders dispersed in petroleum or hydrophilic petroleum containing the active agent can also be suitable. A variety of occlusive devices can be used to release the active agent into the blood stream, such as a semi-permeable membrane covering a reservoir containing the active agent with or without a carrier, or a matrix containing the active agent. Other occlusive devices are known in the literature.

Compounds and/or compositions described herein can be administered rectally or vaginally in the form of a conventional suppository. Suppository formulations can be made from traditional materials, including cocoa butter, with or without the addition of waxes to alter the suppository's melting point and/or glycerin. Water-soluble suppository bases, such as polyethylene glycols of various molecular weights, can also be used.

Lipid formulations or nanocapsules can be used to introduce active agents into host cells either in vitro or in vivo. Lipid formulations and nanocapsules can be prepared by methods known in the art.

To increase the effectiveness of active agents, it can be desirable to combine an active agent with other agents effective in the treatment of the target disease. For example, other active agents effective in treating the target disease can be administered with the active agents. The other agents can be administered at the same time or at different times than the active agents disclosed herein.

Active agents of the invention can be useful for the treatment or inhibition of a pathological condition or disorder in a mammal, for example, a human patient. The invention accordingly provides methods of treating or inhibiting a pathological condition or disorder by providing to a mammal an active agent of the invention.

In certain embodiments, the method is effective to treat a condition associated with hyperproliferating cells, which typically results in a benign or malignant tumor. The method is particularly suitable for treating cancer.

Examples of cancers that are treatable by the method of the invention include, but are not limited to: bone cancer, pancreatic cancer, skin cancer, brain cancer, lung cancer, colorectal cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, multiple myeloma, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

The dosages of the active agents can vary according to, for example, the particular use for which the treatment is made, the manner of administration of the compound, the health and condition of the patient and the judgment of the prescribing physician. The proportion or concentration of a compound of the invention in a pharmaceutical composition can vary depending upon a number of factors including: dosage, chemical characteristics (e.g.: hydrophobicity) and the route of administration. For example, the metal complex and the API can each be provided in an aqueous physiological buffer solution containing about 0.1 to about 10% w/v of the compound for parenteral administration. Some typical dose ranges are from approximately 1 ug/kg to about 1 g/kg of body weight per day. In some embodiments, the dose range is from approximately 0.01 mg/kg to about 100 mg/kg of body weight per day. In certain embodiments, the dosage form comprises each active agent in amounts from about 0.001 mg to about 1000 mg or 0.01 mg to 100 mg or 0.1 mg to 10 mg. The dosage is likely to depend on such variables as the type and extent of progression of the disease or disorder, the overall health status of the particular patient, the relative biological efficacy of the compound selected, formulation of the excipient and its route of administration. Effective doses can be extrapolated from dose-response curves derived from in vitro or in vivo model test systems.

In certain embodiments, the primary therapeutic agent (e.g., an active pharmaceutical ingredient) will be administered systemically either prior to or after the metal complex administration (treatments vary from a few months to a few years). In certain embodiments, the primary therapeutic agent is administered to the patient: locally, intravesically, intratumorally, intravenously or by other administration methods: daily, every 2 days, 3 times per week, every 3 days, weekly, biweekly, monthly, quarterly or some other administration schedule based on the patient's disease, stage and grade in an amount that ranges from the maximum recommended starting dose (MRSD) based on the human equivalent dose (HED) to the therapeutic dose (TD) or biologically effective dose (BED).

In certain embodiments, the metal complex will be administered locally and light activated (1 to 3 treatments) or the metal complex will be administered systemically and activated via radiation (3 treatments per week over 2 to 6 weeks, with radiation daily Monday to Friday for the same time period). In certain embodiments, the metal complex is administered to the patient: locally, intravesically, intratumorally, intravenously or by other administration methods: daily, every 2 days, 3 times per week, every 3 days, weekly, biweekly, monthly, quarterly or some other administration schedule based on the patient's disease, stage and grade in an amount that ranges from the MRSD based on the HED to the TD or BED.

RDT is administered to the patient to activate at least one metal complex present to treat the condition. The term “radiation” as used herein encompasses non-ionizing radiation and ionizing radiation of the electromagnetic spectrum, including: infrared light, visible light, X-rays, Y-rays and quanta, and corpuscular radiation (i.e.: a-particles, p-particles, positrons, neutrons and heavy particles) capable of producing ions, in the less than 180 nanometers (“nm”) range. Suitable wavelengths of light activation; include, but are not limited to 180 to 1000 nm and most preferably 400 to 950 nm.

Radiation is directly ionizing if it carries an electric charge that directly interacts with atoms in the tissue or medium by electrostatic attraction. Indirect ionizing radiation is not electrically charged, but results in production of charged particles by which its energy is absorbed. It takes about 34 eV of energy to produce an ionization. Most human exposures to radiation are of energies of 0.05-5 Million electron Volts (MeV)—energies at which many ionizations occur as the radiation passes through cells. Most X-rays have a wavelength ranging from 0.001 to 10 nanometers. In the case of using a radio enhancer, a patient can be treated with a “diagnostic” dose of ionizing radiation, such as 0.02 Gray (Gy).

The radiation can be applied systemically or locally, topically or internally. The radiation is administered in a safe and effective dosage. For example, laser light is preferably administered in a dosage of at least 10 J/cm², preferably 10 or 100 J/cm² and more preferably from 25 to 90 J/cm². Radiation is preferably administered at a predetermined fluence rate or radiation dose to achieve the most desirable therapeutic effect-up to the highest permissible radiation dose, based on the patient's clinical status.

### Metal-Binding Glycoproteins

The metal complex is preferably administered to the patient along with a metal-binding glycoprotein. Metal-binding glycoproteins suitable for use in the invention are capable of binding transition metals and delivering to a biological target said metals and other materials complexed with said metals. The metal-binding glycoproteins are preferably capable of binding Group 8 metals and/or Group 9 metals, and most preferably Ru, Os and Rh. Most preferred are the iron-binding glycoproteins: transferrin, lactoferrin, ovotransferrin and melanotransferrin and variants thereof, with transferrin being most preferred. The glycoprotein can be purified from natural sources or can be from artificial sources. Thus, for example, the glycoprotein in certain embodiments is a recombinant transferrin, such as Apo Transferrin or OPTIFERRIN, a recombinant human transferrin available from InVitria, a division of Ventria Bioscience.

The invention will be illustrated in more detail with reference to the following examples, but it should be understood that the present invention is not deemed to be limited thereto.

### EXAMPLES

### Example 1: RUTHERRIN Reversing MDR by Potentiating the Effect of Temozolomide Chemotherapy in Human Glioma Cells

Glio Blastoma Multiforme (“GBM”) is the most common malignant primary brain tumor in adults. The alkylating agent Temozolomide (“TMZ”) might prolong overall survival, but resistance evolution represents an important clinical problem. See Stritzelberger et al. (2018). Acquired temozolomide resistance in human glioblastoma cell line U251 is caused by mismatch repair deficiency and can be overcome by lomustine. Clinical & translational oncology: official publication of the Federation of Spanish Oncology Societies and of the National Cancer Institute of Mexico, 20(4), 508-516. https://doi.org/10.1007/s12094-017-1743-x.

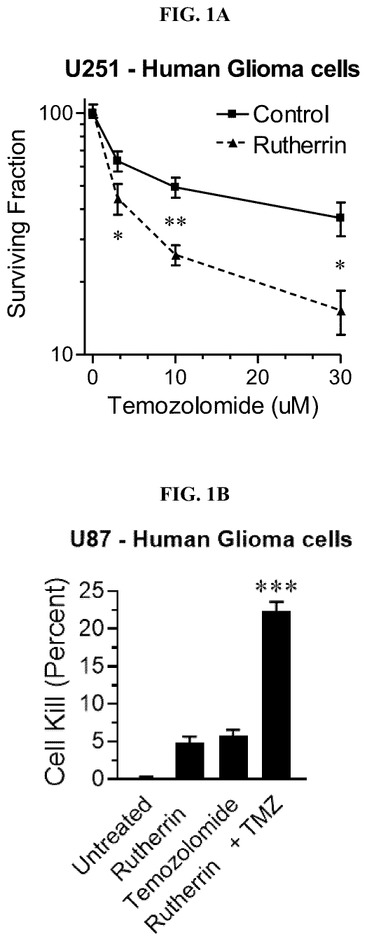

U251 (human glioma) cells were plated overnight, then treated with 3 μM of RUTHERRIN for 4 hours, then treated with increasing doses of the chemotherapeutic agent, Temozolomide, commonly used in combination with radiation therapy of human GBM. The next day the drug was washed, and plates were kept in incubator for 10 days, followed by colony formation assessment and calculation of survival fraction. Sec FIG. 1A.

U87 (another human glioma) cells were plated overnight, then treated with/without 3 μM of RUTHERRIN for 4 hours, followed by 100 μM TMZ overnight. The next day, cell survival was analyzed using presto blue assay. Sec FIG. 1B.

FIGS. 1A and 1B show that RUTHERRIN potentiates the effect of TMZ in human glioma cells and suggests that the combination of RUTHERRIN with the standard of care for GBM (namely chemoradiotherapy) could have a dual effect leading to an increasingly effective therapy.

### Example 2: RUTHERRIN Potentiates the Effect of Multiple Chemotherapeutic Drugs

U87 human glioma cells were plated overnight, then treated with 3 μM of RUTHERRIN for 4 hours, then treated with different chemotherapeutic drugs overnight. Concentrations for the drugs used were as follows: Vandetanib (15 μM), Withaferin A (1.5 μM), Vemurafenib (10 μM), Amiodarone (20 μM), and Vinblatine (20 μM). The next day, cell survival was analyzed using presto blue assay.

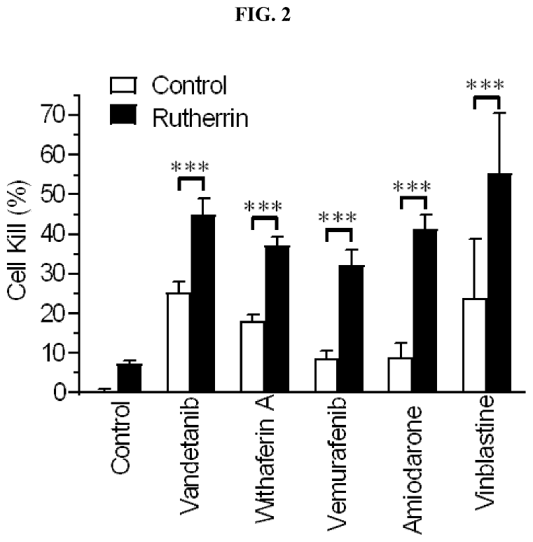

FIG. 2 shows that RUTHERRIN pretreatment significantly potentiates the effect of multiple classes of chemotherapeutic drugs, suggesting a more generalized effect of RUTHERRIN on cancer cells, which renders the cancer cells more susceptible (less resistant to chemotherapy).

### Example 3: RUTHERRIN Potentiates the Effect of VINBLASTINE Treatment in Mouse CT26.WT SQ Model

Chemotherapy, radiotherapy, and molecularly targeted therapy could improve the prognosis of Colorectal Cancer (“CRC”) patients. Recently, immunotherapy, especially immune checkpoint inhibitors, has significantly improved the prognosis of some patients; however, drug resistance significantly reduces the usefulness of these drugs. Although research on the molecular mechanisms underlying the emergence of drug resistance is ongoing, the specific molecular mechanisms remain unclear.

Balb/c mice were injected with CT26.WT colorectal cancer cells Subcutaneous (“SQ”) at day 0. Mice were injected with RUTHERRIN (10 mg/kg) three times per week (Monday, Wednesday, and Friday), and with Vinblastine (0.75 mg/kg) twice in the first week, then weekly afterwards, until endpoints were reached. Mice were monitored through tumor measurements in 3 dimensions to calculate tumor volume, and endpoints were reached when tumor size reached more than 1.5 cm in length.

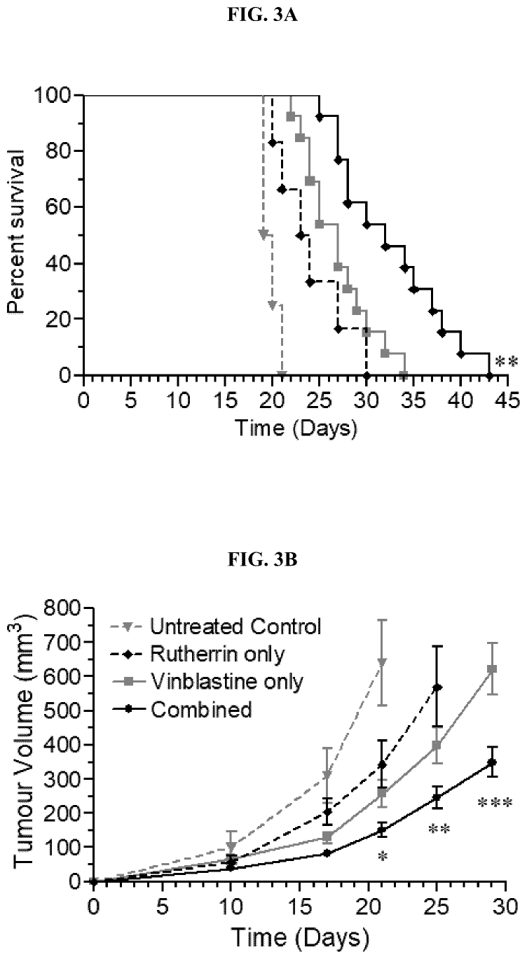

FIG. 3A shows the resulting Kaplan-Meier survival analysis. FIG. 3B shows the resulting tumor volume quantification over time. The graphs show an effect of RUTHERRIN alone treatment on tumor volume and survival, and when combined with Vinblastine, there is a significant reduction in tumor volume progression, resulting in improved survival.

### Example 4: RUTHERRIN Pre-Treatment Results in Increased Drug Retention in Cancer Cells

Cancer cells are known to be resistant to chemotherapy through multiple mechanisms, including the inactivation of the drug, altering drug metabolism, inhibition of cell death (apoptosis suppression), epigenetic changes, changes in the drug targets, enhancing DNA repair mechanisms, and reducing intracellular drug accumulation by increasing drug efflux from the cell via energy-dependent efflux pumps. The latter is the main mechanism of cancer resistance to chemotherapy.

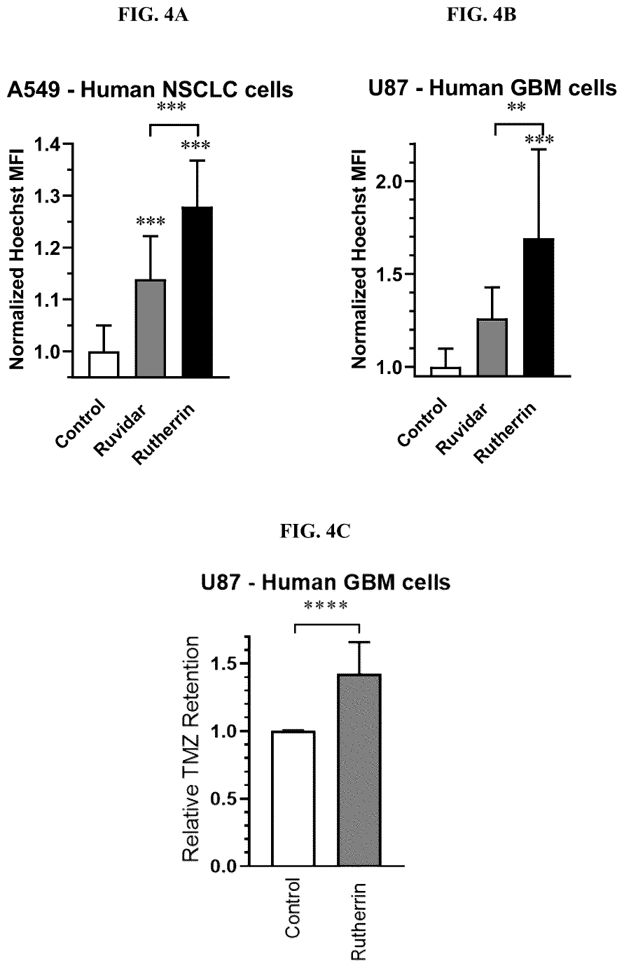

Hoechst 33342 is a nuclear dye commonly used to quantify drug efflux dynamics and drug accumulation in cells. In FIGS. 4A and 4B, U87 human glioma cells or A549 human Non-Small Cell Lung Cancer (“NSCLC”) cells were plated overnight, then treated with 3 μM of RUVIDAR or RUTHERRIN for 4 hours, then incubated with Hoescht 33342 for 1 hour. Cells were then collected, and the MFI was determined by flow cytometry. FIGS. 4A and 4B show that RUVIDAR (and significantly more RUTHERRIN) treatment increases dye accumulation inside the cells, possibly through inhibition of efflux channels.

The observation was confirmed using the chemotherapeutic drug, TMZ, in U87 human glioma cells. Cells were plated overnight, then treated with 3 μM RUTHERRIN for 4 hours, then treated with 100 μM TMZ for 2 hours. Cells were then collected and sonicated to disrupt cells and release intracellular drug. Samples were then centrifuged and dried using ethyl acetate, then reconstituted in methanol. TMZ concentration was determined through HPLC-MS and Theophylline was used as an internal standard. The concentration of TMZ was normalized to the internal standard in each sample, and RUTHERRIN treated samples were normalized to TMZ only control. FIG. 4C shows increased TMZ accumulation in RUTHERRIN pretreated cells, suggesting a possible mechanism that could explain the increase in TMZ efficacy observed in Example 1.

### Example 5: effect of RUTHERRIN on Metformin Cell Kill

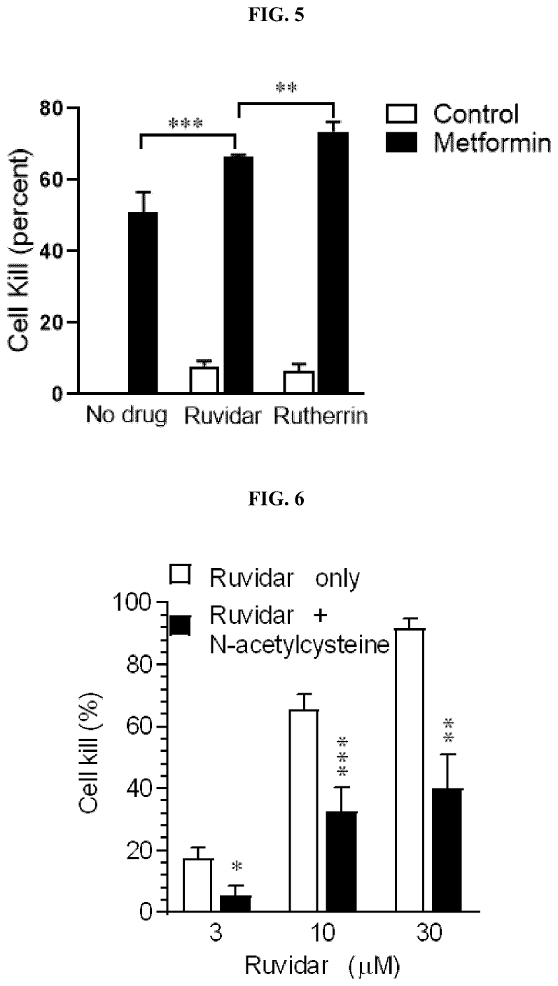

FIG. 5 shows the results of T24 human bladder cancer cells being plated overnight, then treated with or without 3 μM RUVIDAR or RUTHERRIN for 4 hours, then treated with (black bars) or without (white bars) 32 mM of Metformin for 48 hours. Cell survival was analyzed using presto blue assay.

FIG. 5 shows that RUTHERRIN pretreatment significantly potentiates the cell kill effects of Metformin, with RUTHERRIN showing a significantly higher effect than RUVIDAR pre-treatment.

### Example 6: RUTHERRIN-Mediated Increase in ROS Production as a Possible Mechanism of Action to Combat MDR

In cancer cells, due to aerobic glycolysis (“Warburg effect”), the level of Reactive Oxygen Species (“ROS”) is higher than normal cells, but is still below the threshold for cytotoxicity. RUVIDAR, which contains a Ruthenium molecule and other ligands, may be activated by the higher redox/oxidative potential in cancer cells, which would in turn cause a further increase in ROS production, and may yield a dose-dependent cytotoxicity of the cancer cell.

U87 human glioma cancer cells were plated overnight, then treated with different doses of RUVIDAR in the presence or absence of N-acetylcysteine, a known ROS scavenger to quench the increase in ROS production. Cells were incubated overnight, and cell survival was analyzed using Presto Blue assay.

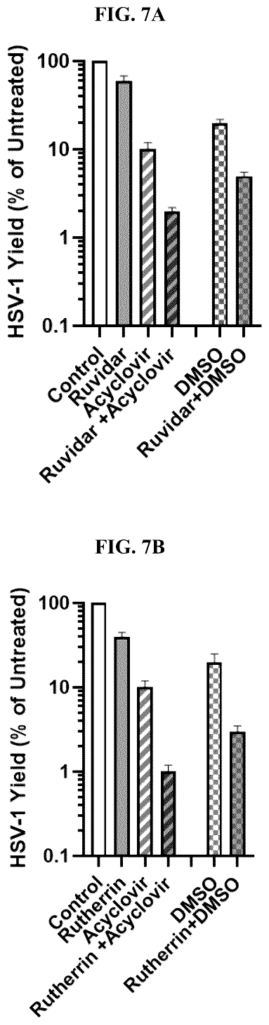

FIG. 6 shows that RUVIDAR only treatment caused a dose-dependent increase in cell kill, which is likely mediated by an increase in ROS production in cells, since the cell kill was significantly reduced in the presence of the ROS scavenger N-acetylcysteine.

### Example 7: RUVIDAR and RUTHERRIN Potentiate the Anti-Viral Effects of Acyclovir and DMSO (Prophetic)

A549 human lung cancer cells are used as a host for virus growth, treatment, and later survival quantification. A549 cells are plated overnight, then incubated for 4 hours with or without 50 nM RUVIDAR or RUTHERRIN, with or without 1 μM Acyclovir or 0.2% DMSO (concentration non-toxic for the host cells). After the initial incubation of the host cells with the compounds, the cells are infected with the human Herpes Simplex 1 (HSV-1) virus at the multiplicity of infection (MOI)=0.3 and further incubated for three days (the mentioned compounds are still present in the culture media over the full course of incubation). After that, supernatant is harvested, centrifuged to remove debris, and the yield of infectious virus is determined by plaque forming assay (PFA) using supernatant for the host cells infection. The formed plaques are counted, and virus titer is calculated as Plaque Forming Units/mL=(Plaques number*Dilution)/Volume of Virus Inoculum. Titers normalized for control titer (untreated HSV-1 and host cells) produce relative virus yield (which is equivalent to inhibition rate).

The data are presented as virus yield normalized to Control. To estimate synergistic effect of two combined compounds, synergy Bliss score was calculated, as the difference between the actual inhibiting effect of the combination and the predicted inhibiting effect under “no synergy” assumption (Bliss score): Synergy score=Inhibition_Rate_{Compound1} & _{Compound2}−(Inhibition_Rate_{Compound1}+Inhibition_Rate_{Compound2}−Inhibition_Rate_{Compound1}* Inhibition_Rate_{Compound2}).

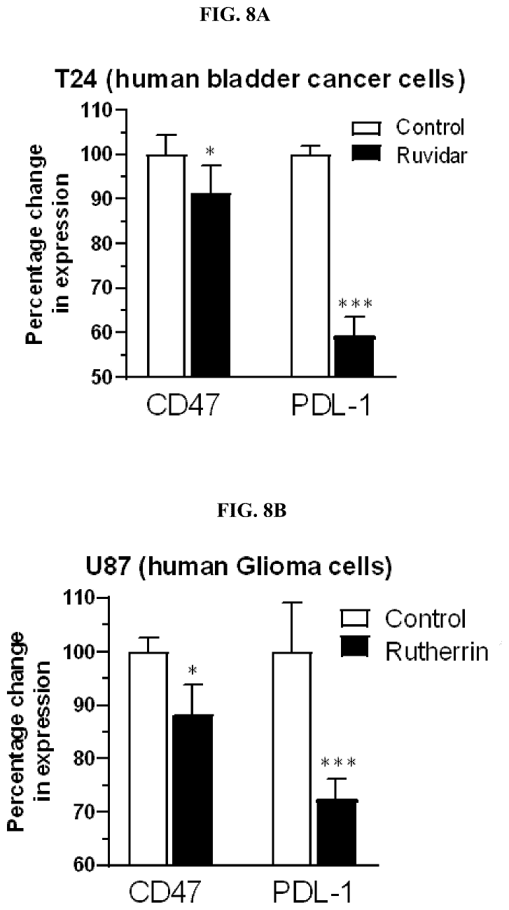

FIG. 7 shows that RUVIDAR, RUTHERRIN, Acyclovir, and DMSO having an anti-viral effect to various degrees and suppressing the yield of HSV-1 propagated in host cells. However, HSV-1 yield suppression is significantly potentiated if RUVIDAR or RUTHERRIN were combined with Acyclovir, with synergistic effect of the mix (synergy score=0.040±0.013, P=0.037, N=5 and 0.030±0.010 P=0.040, respectively), or with DMSO (P<0.0001), also synergistically (synergy score=0.030±0.010, P=0.040, and 0.050±0.015, P=0.029, respectively). The data demonstrate that RUVIDAR and RUTHERRIN can considerably potentiate the effect of established anti-viral drugs.

### Example 8: RUVIDAR and RUTHERRIN Downregulates the Expression of Immune Checkpoint Proteins

Among the hallmarks of cancer resistance is the overexpression of certain proteins that help cancer cells evade the host's immune system surveillance. CD47 is expressed in many normal cells to tell circulating macrophages not to eat these cells, but it is overexpressed by cancer cells to evade macrophage destruction. PD-L1 is another protein expressed on cells to prevent T cells from killing the PD-L1 expressing cells, and this marker is also overexpressed by cancer cells to evade T cell destruction. It was also reported that cancer cells express high levels of transferrin receptors, and it correlates with high expression of PD-L1, CD47, and CTLA4 to inhibit the function antigen presenting cells and the recruitment and activation of T cells.

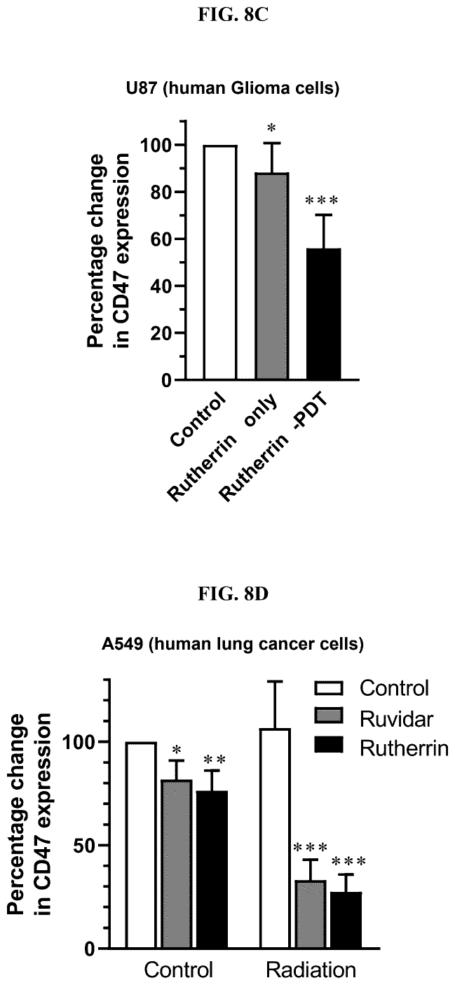

In the tests whose results are shown in FIGS. 8A and 8B, T24 human bladder cancer cells or U87 human glioma cells were treated with 3 μM of RUVIDAR or RUTHERRIN for 2 days, and cells were collected and stained with fluorescent-labelled antibodies to CD47 and PD-L1 and analyzed by flow cytometry. MFI was normalized to control untreated cells and expressed as percentage of change. RUVIDAR or RUTHERRIN treatment only significantly reduced the expression of both CD47 and PD-L1 in two different cell lines, suggesting that drug treatment makes cancer cell more sensitive to immune recognition, which would facilitate the activation of the immune response.

In the tests whose results are shown in FIGS. 8C and 8D, U87 human glioma cells or A549 lung cancer cells were treated with 3 μM of RUTHERRIN or RUVIDAR prior to drug activation by 530 nm laser light (photodynamic therapy, PDT) or ionizing radiation. Cells were then collected and stained with fluorescent antibody to CD47 and analyzed by flow cytometry. MFI was normalized to control untreated cells and expressed as percentage of change. While drug alone treatment significantly reduced the expression of CD47, the combination with either PDT or ionizing radiation resulted in a more significant reduction in CD47 expression, while ionizing radiation alone did not have any effect on CD47 expression, suggesting that combination of RUTHERRIN or RUVIDAR with ionizing radiation may make cancer cells more sensitive to immune recognition and activation of an immune response compared to radiation alone.

### Example 9: RUTHERRIN-Induced Retention of Cisplatin or Gemcitabine in Cancer Cells

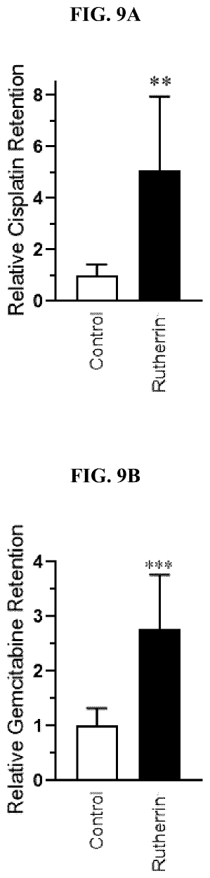

Human A549 lung cancer cells were plated overnight, then treated with 3 μM RUTHERRIN for 4 hours, then treated with 125 ug/ml Cisplatin for 40 minutes. Cells were then collected and sonicated to disrupt cells and release intracellular drug. Platinum was derivatized using incubation with diethyldithiocarbamate (DDTC) for 45 minutes at 37° C. Palladium acetate was added as a derivatization control. Samples were then centrifuged and dried using acetonitrile. Cisplatin concentration was determined through HPLC-MS. 8-cyclopentyl-1,3-dipropylxanthine (DPCPX) was used as an internal standard. The concentration of Cisplatin was normalized to the internal standard and derivatization standard in each sample, and RUTHERRIN treated samples were normalized to the Cisplatin only control. FIG. 9A shows increased Cisplatin accumulation in RUTHERRIN pretreated cells.

Human T24 bladder cancer cells were plated overnight, then treated with 3 uM RUTHERRIN for 4 hours, then treated with 30 ug/ml Gemcitabine for 40 minutes. Cells were then collected in Acetonitrile: Methanol:Water (40:40:20) and sonicated to disrupt cells and release intracellular drug. Samples were then centrifuged and dried using speed vacuum. Gemcitabine concentration was determined through HPLC-MS. Gemcitabine (13C,15N2) was used as an internal standard. The concentration of Gemcitabine was normalized to the internal standard in each sample, and RUTHERRIN treated samples were normalized to the Cisplatin only control. FIG. 9B shows increased Gemcitabine accumulation in RUTHERRIN pretreated cells.

### Example 10: RUTHERRIN Potentiates the Cell Kill Effect of Cisplatin and Gemcitabine in Cancer Cells

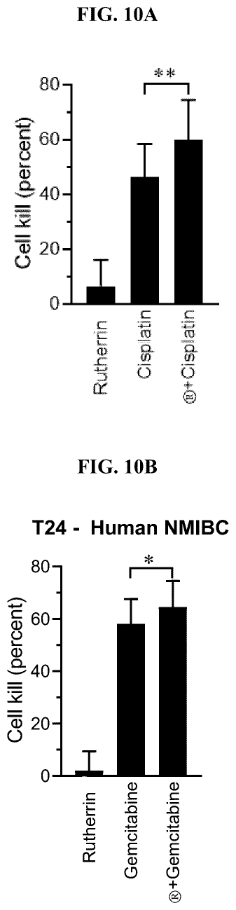

Cells were plated overnight, then treated with 3 uM RUTHERRIN for 4 hours, then treated with 3 ug/ml Cisplatin or 10 ng/ml Gemcitabine for 48 hours. Cell viability was determined by Presto Blue assay and was normalized to control untreated cells. FIGS. 10A and 10B show that RUTHERRIN pretreatment potentiates cisplatin or gemcitabine cancer cell killing compared to either RUTHERRIN or cisplatin/gemcitabine alone, potentially because of increase drug retention in cancer cells.

### Additional Numbered Embodiments of the Invention

1. A method for treating a condition, said method comprising the steps of: selecting a patient for treatment who has the condition; administering to the patient at least one active pharmaceutical ingredient (API); and administering to the patient at least one metal complex represented by formula (I):

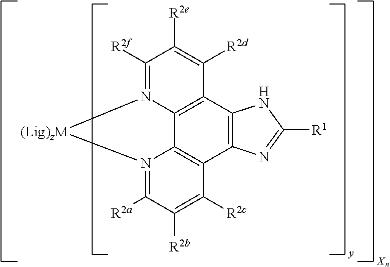

including hydrates, solvates, pharmaceutically acceptable salts and prodrugs thereof, wherein:
M is selected from the group consisting of manganese, molybdenum, rhenium, iron, ruthenium, osmium, cobalt, rhodium, iridium, nickel, platinum, and copper;
X is selected from the group consisting of Cl⁻, PF₆⁻, Br⁻, BF₄⁻, ClO₄⁻, CF₃SO₃⁻, and SO₄⁻².
n=0, 1, 2, 3, 4, or 5;
y=1, 2, or 3;
z=0, 1, or 2;
Lig at each occurrence is independently selected from the group consisting of

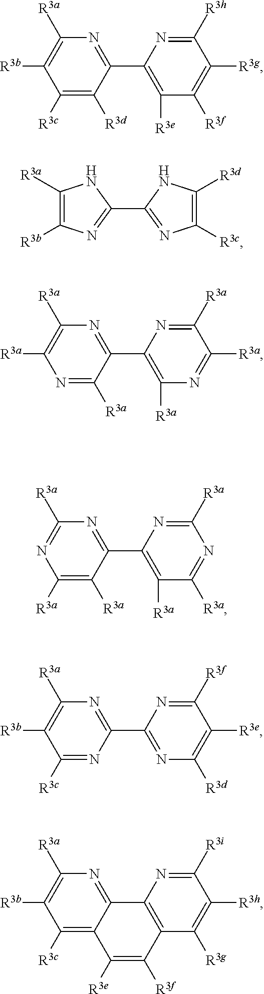

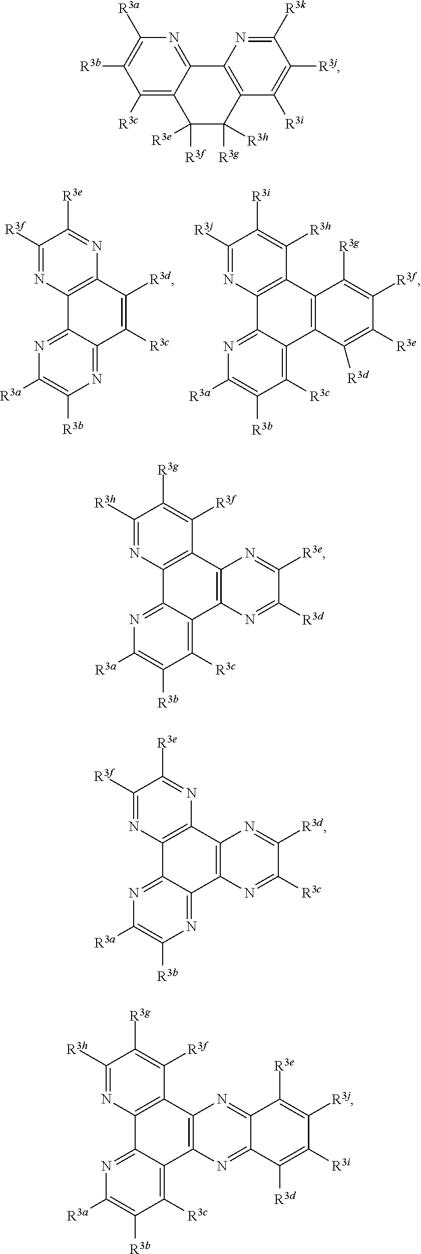

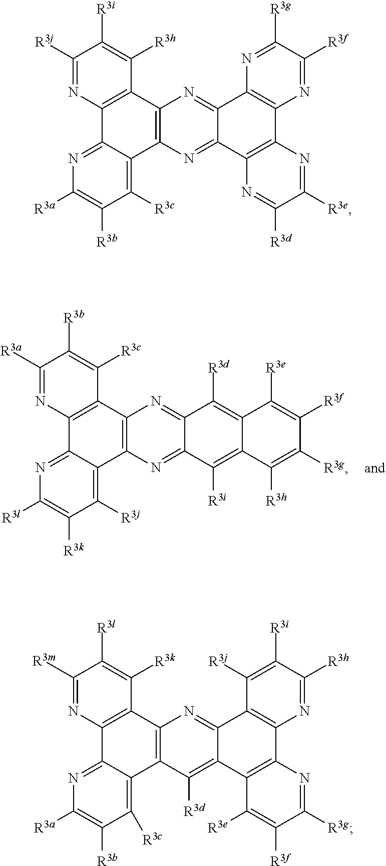

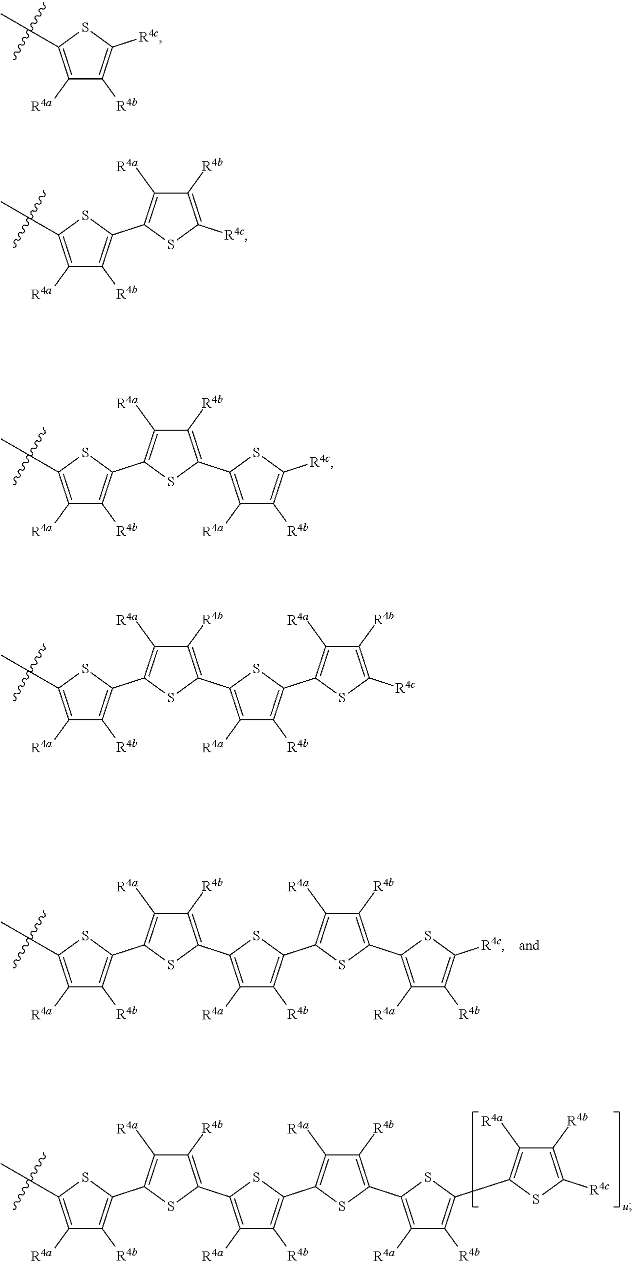

R′ is selected from the group consisting of
u is an integer of 1 to 20;
R^{2a}, R^{2b}, R^{2c}, R^{2d}, R^{2e}, and R^{2f} at each occurrence are each independently selected from the group consisting of hydrogen, C1-6 optionally substituted alkyl, C1-6 optionally substituted branched alkyl, C3-7 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h} R³ⁱ, R^{3j}, R^{3k}, R^{3l}, and R^{3m} at each occurrence are each independently selected from the group consisting of hydrogen, C1-6 optionally substituted alkyl, C1-6 optionally substituted branched alkyl, C1-6 optionally substituted haloalkyl, C1-6 optionally substituted alkoxy, and CO₂R⁸;
R^{4a}, R^{4b}, and R^{4c} at each occurrence are each independently selected from the group consisting of hydrogen, C1-6 optionally substituted alkyl, C1-6 optionally substituted branched alkyl, C1-6 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence is independently selected from the group consisting of hydrogen and optionally substituted alkyl;
R⁶ at each occurrence is independently selected from the group consisting of hydrogen and optionally substituted alkyl;
R⁷ at each occurrence is independently selected from the group consisting of hydrogen and optionally substituted alkyl; and
R⁸ at each occurrence is independently selected from the group consisting of hydrogen and optionally substituted alkyl,
wherein:
the administering to the patient of the at least one metal complex is conducted prior to, during and/or after the administering to the patient of the at least one API; and the at least one API has a structure not represented by Formula (I).
2. The method of embodiment 1, wherein the administering to the patient of the at least one metal complex causes an improvement in an efficacy of treating the condition relative to continued treatment of the condition with only the at least one API.
3. The method of embodiment 1 or 2, wherein the at least one metal complex is administered to the patient only after the condition has developed a resistance to the at least one API, which continues to be administered to the patient along with the at least one metal complex.
4. The method of embodiment 1 or 2, wherein the at least one metal complex is administered to the patient prior to or concurrently with the step of administering to the patient the at least one active pharmaceutical ingredient.
5. The method of any one of embodiments 1-4, wherein the condition is cancer, and the at least one API is at least one member selected from the group consisting of a chemotherapeutic agent, an immunotherapeutic agent and a targeted therapeutic agent.
6. The method of any one of embodiments 1-4, wherein the condition is an infectious disease and the at least one API is at least one member selected from the group consisting of an antibiotic agent, an antimicrobial agent, an antifungal agent and an antiviral agent.
7. The method of any one of embodiments 1-6, wherein the at least one API is selected from the group consisting of Cisplatin, Temozolomide, Vandetanib, Withaferin A, Vemurafenib, Amiodarone, Vinblastine, Metformin, Gemcitabine and Acyclovir.
8. The method of any one of embodiments 1-7, further comprising administering transferrin to the patient.
9. The method of any one of embodiments 1-8, further comprising administering to the patient radiation selected from the group consisting of infrared light, visible light, X-rays and gamma rays.
10. The method of any one of embodiments 1-9, wherein the at least one metal complex comprises at least one member selected from the group consisting of:
Ru(2,2′-bipyridine)₂(2-(2′,2″:5″,2″′-terthiophene)-imidazo[4,5-f][1,10] phenanthroline);
Ru(2,2′-bipyridine)₂(2-(2′,2″:5″,2″′;5″′,2″″quaterthiophene)-imidazo[4,5-f][1,10] phenanthroline);
Ru(4,4′-dimethyl-2,2′-bipyridine)₂(2-thiophenimidazo[4,5-f][1,10] phenanthroline);
Ru(4,4′-dimethyl-2,2′-bipyridine)₂(2-(2′,2″-bithiophene)-imidazo[4,5-f][1,10] phenanthroline);
Ru(4,4′-dimethyl-2,2′-bipyridine)₂(2-(2′,2″:5″,2′″-terthiophene)-imidazo[4,5-f][1,10] phenanthroline);
Ru(4,4′-dimethyl-2,2′-bipyridine)₂(2-(2′,2″:5″,2″′;5″′,2″″-quaterthiophene) -imidazo[4,5-f][1,10] phenanthroline);
Ru(4,4′-di-t-butyl-2,2′-bipyridine)₂(2-thiophenimidazo[4,5-f][1,10] phenanthroline); Ru(4,4′-di-t-butyl-2,2′-bipyridine)₂(2-(2′,2″-bithiophene)-imidazo[4,5 -f][1,10] phenanthroline);
Ru(4,4′-di-t-butyl-2,2′-bipyridine)₂(2-(2′,2″:5″,2′″-terthiophene)-imidazo[4,5-f][1,10] phenanthroline);
Ru(4,4′-dimethoxy-2,2′-bipyridine)₂(2-(2′,2″:5″,2′″-terthiophene)-imidazo[4,5-f][1,10] phenanthroline);
Ru(5,5′-dimethyl-2,2′-bipyridine)₂(2-thiophenimidazo[4,5-f][1,10] phenanthroline);
Ru(5,5′-dimethyl-2,2′-bipyridine)₂(2-(2′,2″-bithiophene)-imidazo[4,5-f][1,10] phenanthroline);
Ru(5,5′-dimethyl-2,2′-bipyridine)₂(2-(2′,2″:5″,2′″-terthiophene)-imidazo[4,5-f][1,10] phenanthroline);
Ru(6,6′-dimethyl-2,2′-bipyridine)₂(2-thiophenimidazo[4,5-f][1,10] phenanthroline);
Ru(6,6′-dimethyl-2,2′-bipyridine)₂(2-(2′,2″-bithiophene)-imidazo[4,5-f][1,10] phenanthroline);
Ru(6,6′-dimethyl-2,2′-bipyridine)₂(2-(2′,2″:5″,2′″-terthiophene)-imidazo[4,5-f][1,10] phenanthroline);
Ru(4,4′-di (methylcarboxy)-2,2′-bipyridine)₂(2-(2′,2″:5″,2′″-terthiophene) -imidazo[4,5-f][1,10] phenanthroline);
Ru(2,2′-bipyrimidine)₂(2-(2′,2″:5″,2″′-terthiophene)-imidazo[4,5-f][1,10] phenanthroline);
Ru(2,2′-bipyrimidine)(4,4′-dimethyl-2,2′-bipyridine)(2-(2′,2″:5″,2″′-terthiophene)-imidazo[4,5-f][1,10] phenanthroline);
Ru(1,10-phenanthroline)₂(2-thiophenimidazo[4,5-f][1,10] phenanthroline);
Os(2,2′-bipyridine)₂(2-thiophenimidazo[4,5-f][1,10] phenanthroline);
Os(2,2′-bipyridine)₂(2-(2′,2″-bithiophene)-imidazo[4,5-f][1,10] phenanthroline);
Os(2,2′-bipyridine)₂(2-(2′,2″:5″,2′″-terthiophene)-imidazo[4,5-f][1,10] phenanthroline);
Os(1,10-phenanthroline)₂(2-thiophenimidazo[4,5-f][1,10] phenanthroline);
Os(1,10-phenanthroline)₂(2-(2′,2″-bithiophene)-imidazo[4,5-f][1,10] phenanthroline);
Os(1,10-phenanthroline)₂(2-(2′,2″:5″,2″′-terthiophene)-imidazo[4,5-f][1,10] phenanthroline);
Os(4,4′-dimethyl-2,2′-bipyridine)₂(2-thiophenimidazo[4,5-f][1,10] phenanthroline);
Os(4,4′-dimethyl-2,2′-bipyridine)₂(2-(2′,2″-bithiophene)-imidazo[4,5-f][1,10] phenanthroline);
Os(4,4′-dimethyl-2,2′-bipyridine)₂(2-(2′,2″:5″,2″′-terthiophene)-imidazo[4,5-f][1,10] phenanthroline); and pharmaceutically acceptable salts thereof.
   11. The method of any one of embodiments 1-9, wherein the at least one metal complex is Ru(4,4′-dimethyl-2,2′-bipyridine)₂(2-(2′,2″:5″,2′-terthiophene)-imidazo[4,5-f][1,10] phenanthroline) or a pharmaceutically acceptable salt thereof.
   12. A composition for combination therapy effective to treat a condition in a patient, said composition comprising:
   at least one active pharmaceutical ingredient (API) selected from the group consisting of Cisplatin, Temozolomide, Vandetanib, Withaferin A, Vemurafenib, Amiodarone, Vinblastine, Metformin, Gemcitabine and Acyclovir; and
   at least one metal complex effective to potentiate an efficacy of the active pharmaceutical ingredient to treat the condition, wherein the at least one metal complex is represented by formula (I):

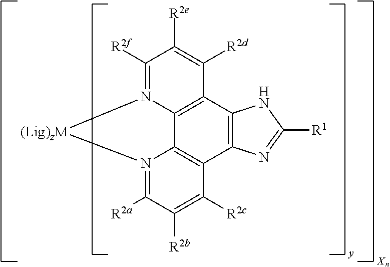

including hydrates, solvates, pharmaceutically acceptable salts and prodrugs thereof, wherein:
M is selected from the group consisting of manganese, molybdenum, rhenium, iron, ruthenium, osmium, cobalt, rhodium, iridium, nickel, platinum, and copper;
X is selected from the group consisting of Cl⁻, PF₆⁻, Br⁻, BF₄⁻, ClO₄⁻, CF₃SO₃⁻, and SO₄⁻²;
n=0, 1, 2, 3, 4, or 5;
y=1, 2, or 3;
z=0, 1, or 2;
Lig at each occurrence is independently selected from the group consisting of

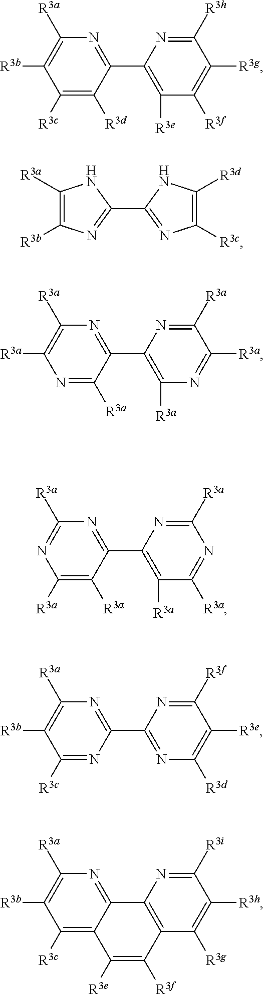

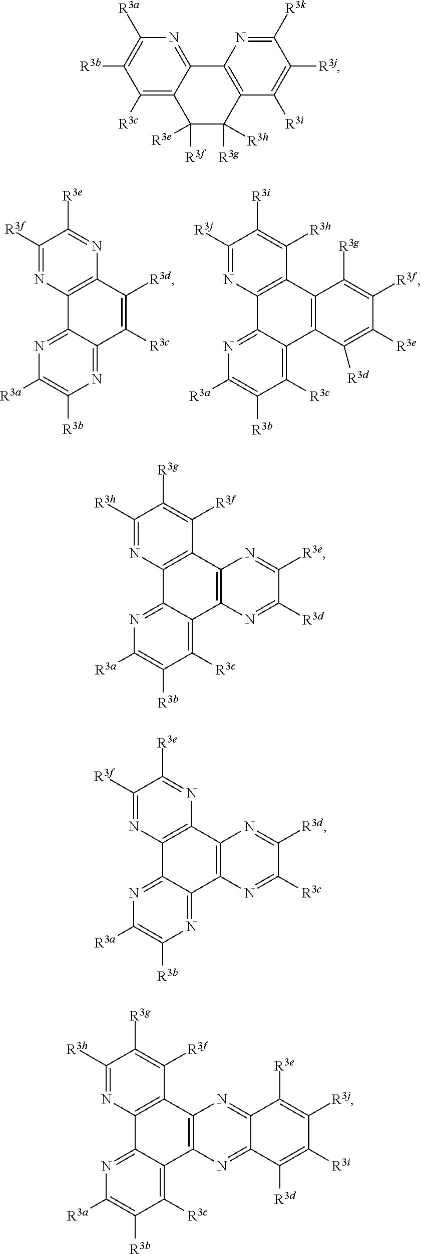

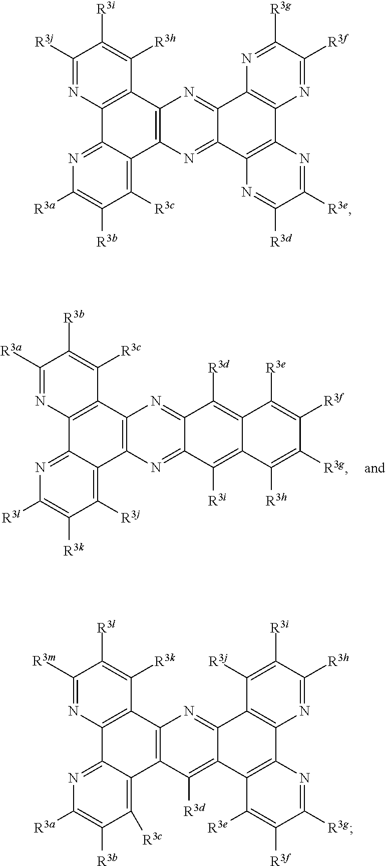

R¹ is selected from the group consisting of.

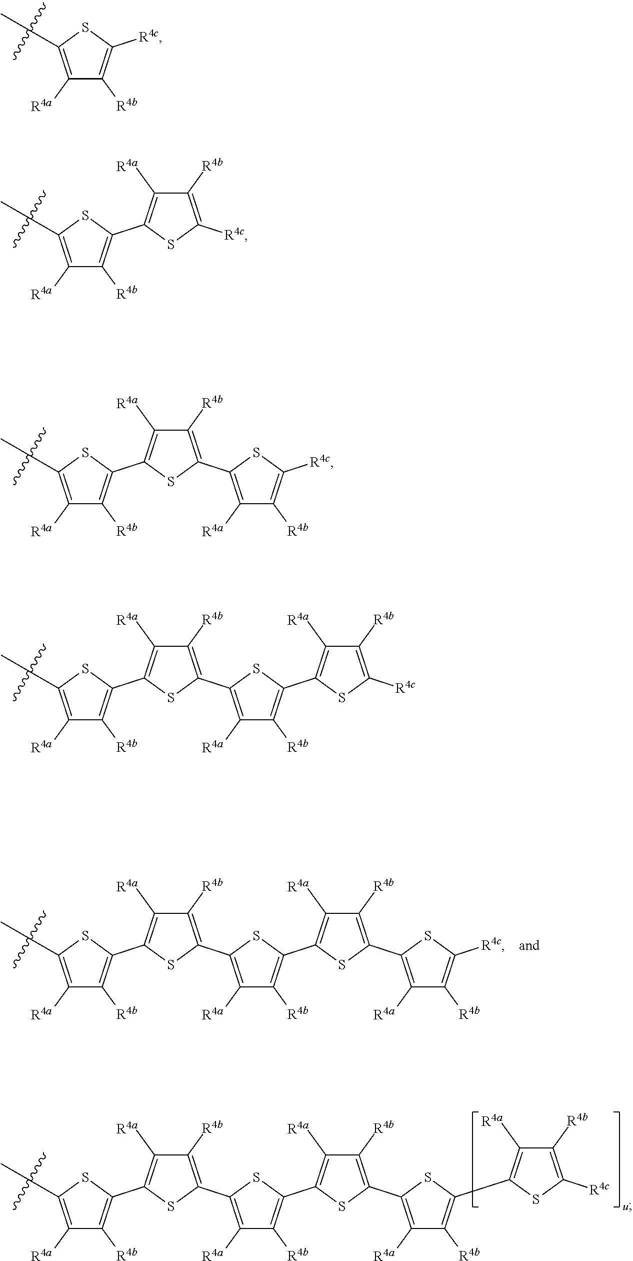

u is an integer of 1 to 20;
R^{2a}, R^{2b}, R^{2c}, R^{2d}, R^{2e}, and R^{2f} at each occurrence are each independently selected from the group consisting of hydrogen, C1-6 optionally substituted alkyl, C1-6 optionally substituted branched alkyl, C3-7 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h} R³ⁱ, R^{3j}, R^{3k}, R^{3l}, and R^{3m} at each occurrence are each independently selected from the group consisting of hydrogen, C1-6 optionally substituted alkyl, C1-6 optionally substituted branched alkyl, C1-6 optionally substituted haloalkyl, C1-6 optionally substituted alkoxy, and CO₂R⁸;
R^{4a}, R^{4b}, and R^{4c} at each occurrence are each independently selected from the group consisting of hydrogen, C1-6 optionally substituted alkyl, C1-6 optionally substituted branched alkyl, C1-6 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence is independently selected from the group consisting of hydrogen and optionally substituted alkyl;

R⁶ at each occurrence is independently selected from the group consisting of hydrogen and optionally substituted alkyl;

R⁷ at each occurrence is independently selected from the group consisting of hydrogen and optionally substituted alkyl; and

R⁸ at each occurrence is independently selected from the group consisting of hydrogen and optionally substituted alkyl.
13. The composition of embodiment 12, wherein the condition is cancer.
14. The composition of embodiment 12, wherein the condition is an infectious disease.
15. The composition of any one of embodiments 12-14, further comprising transferrin.
16. The composition of any one of embodiments 12-15, wherein the at least one metal complex comprises at least one member selected from the group consisting of:
Ru(2,2′-bipyridine)₂(2-(2′,2″:5″,2′″-terthiophene)-imidazo[4,5-f][1,10] phenanthroline);
Ru(2,2′-bipyridine)₂(2-(2′,2″:5″,2″′;5″′,2″″-quaterthiophene)-imidazo[4,5-f][1,10] phenanthroline);
Ru(4,4′-dimethyl-2,2′-bipyridine)₂(2-thiophenimidazo[4,5-f][1,10] phenanthroline);
Ru(4,4′-dimethyl-2,2′-bipyridine)₂(2-(2′,2″-bithiophene)-imidazo[4,5- f][1,10] phenanthroline);
Ru(4,4′-dimethyl-2,2′-bipyridine)₂(2-(2′,2″:5″,2′″-terthiophene)-imidazo[4,5-f][1,10] phenanthroline);
Ru(4,4′-dimethyl-2,2′-bipyridine)₂(2-(2′,2″:5″,2″′;5′″,2″″-quaterthiophene) -imidazo[4,5-f][1,10] phenanthroline);
Ru(4,4′-di-t-butyl-2,2′-bipyridine)₂(2-thiophenimidazo[4,5-f][1,10] phenanthroline);
Ru(4,4′-di-t-butyl-2,2′-bipyridine)₂(2-(2′,2″-bithiophene)-imidazo[4,5-f][1,10] phenanthroline);
Ru(4,4′-di-t-butyl-2,2′-bipyridine)₂(2-(2′,2″:5″,2′″-terthiophene)-imidazo[4,5-f][1,10] phenanthroline);
Ru(4,4′-dimethoxy-2,2′-bipyridine)₂(2-(2′,2″:5″,2″′-terthiophene)-imidazo[4,5-f][1,10] phenanthroline);
Ru(5,5′-dimethyl-2,2′-bipyridine)₂(2-thiophenimidazo[4,5-f][1,10] phenanthroline);
Ru(5,5′-dimethyl-2,2′-bipyridine)₂(2-(2′,2″-bithiophene)-imidazo[4,5-f][1,10] phenanthroline);
Ru(5,5′-dimethyl-2,2′-bipyridine)₂(2-(2′,2″:5″,2′″-terthiophene)-imidazo[4,5-f][1,10] phenanthroline);
Ru(6,6′-dimethyl-2,2′-bipyridine)₂(2-thiophenimidazo[4,5-f][1,10] phenanthroline); Ru(6,6′-dimethyl-2,2′-bipyridine)₂(2-(2′,2″-bithiophene)-imidazo[4,5-f][1,10] phenanthroline);
Ru(6,6′-dimethyl-2,2′-bipyridine)₂(2-(2′,2″:5″,2′″-terthiophene)-imidazo[4,5-f][1,10] phenanthroline);
Ru(4,4′-di (methylcarboxy)-2,2′-bipyridine)₂(2-(2′,2″:5″,2′″-terthiophene) -imidazo[4,5-f][1,10] phenanthroline);
Ru(2,2′-bipyrimidine)₂(2-(2′,2″:5″,2″′-terthiophene)-imidazo[4,5-f][1,10] phenanthroline);
Ru(2,2′-bipyrimidine)(4,4′-dimethyl-2,2′-bipyridine)(2-(2′,2″:5″,2″′-terthiophene)-imidazo[4,5-f][1,10] phenanthroline);
Ru(1,10-phenanthroline)₂(2-thiophenimidazo[4,5-f][1,10] phenanthroline);
Os(2,2′-bipyridine)₂(2-thiophenimidazo[4,5-f][1,10] phenanthroline);
Os(2,2′-bipyridine)₂(2-(2′,2″-bithiophene)-imidazo[4,5-f][1,10] phenanthroline);
Os(2,2′-bipyridine)₂(2-(2′,2″:5″,2′″-terthiophene)-imidazo[4,5-f][1,10] phenanthroline);
Os(1,10-phenanthroline)₂(2-thiophenimidazo[4,5-f][1,10] phenanthroline);
Os(1,10-phenanthroline)₂(2-(2′,2″-bithiophene)-imidazo[4,5-f][1,10] phenanthroline);
Os(1,10-phenanthroline)₂(2-(2′,2″:5″,2′″-terthiophene)-imidazo[4,5-f][1,10] phenanthroline);
Os(4,4′-dimethyl-2,2′-bipyridine)₂(2-thiophenimidazo[4,5-f][1,10] phenanthroline);
Os(4,4′-dimethyl-2,2′-bipyridine)₂(2-(2′,2″-bithiophene)-imidazo[4,5-f][1,10] phenanthroline);
Os(4,4′-dimethyl-2,2′-bipyridine)₂(2-(2′,2″:5″,2′″-terthiophene)-imidazo[4,5-f][1,10] phenanthroline); and pharmaceutically acceptable salts thereof.
   17. The composition of any one of embodiments 12-15, wherein the at least one metal complex is Ru(4,4′-dimethyl-2,2′-bipyridine)₂(2-(2′,2″:5″,2″-terthiophene) -imidazo[4,5-f][1,10] phenanthroline) or a pharmaceutically acceptable salt thereof.

While the invention has been described in detail and with reference to specific examples thereof, it will be apparent to one skilled in the art that various changes and modifications can be made therein without departing from the spirit and scope thereof.

What is claimed is:

1. A method for treating a condition, said method comprising the steps of:
selecting a patient for treatment who has the condition;
administering to the patient at least one active pharmaceutical ingredient (API); and
administering to the patient at least one metal complex represented by formula (I): 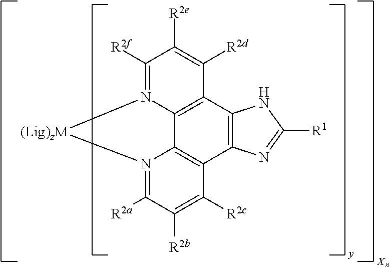
including hydrates, solvates, pharmaceutically acceptable salts and prodrugs thereof, wherein:
M is selected from the group consisting of manganese, molybdenum, rhenium, iron, ruthenium, osmium, cobalt, rhodium, iridium, nickel, platinum, and copper;
X is selected from the group consisting of Cl⁻, PF₆⁻, Br⁻, BF₄⁻, ClO₄⁻, CF₃SO₃⁻, and SO₄⁻².
n=0, 1, 2, 3, 4, or 5;
y=1, 2, or 3;
z=0, 1, or 2;
Lig at each occurrence is independently selected from the group consisting of 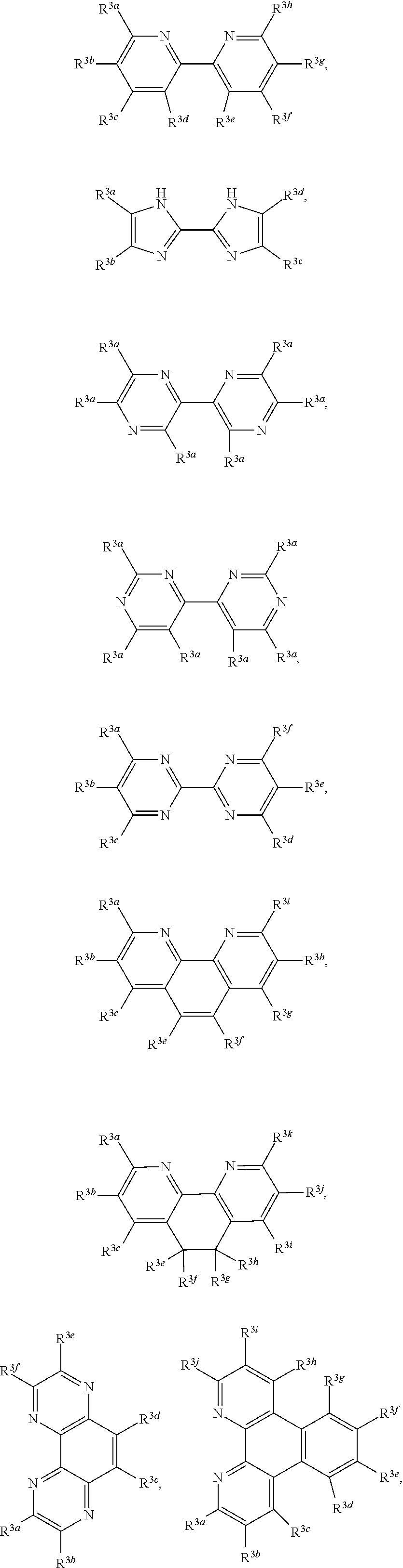 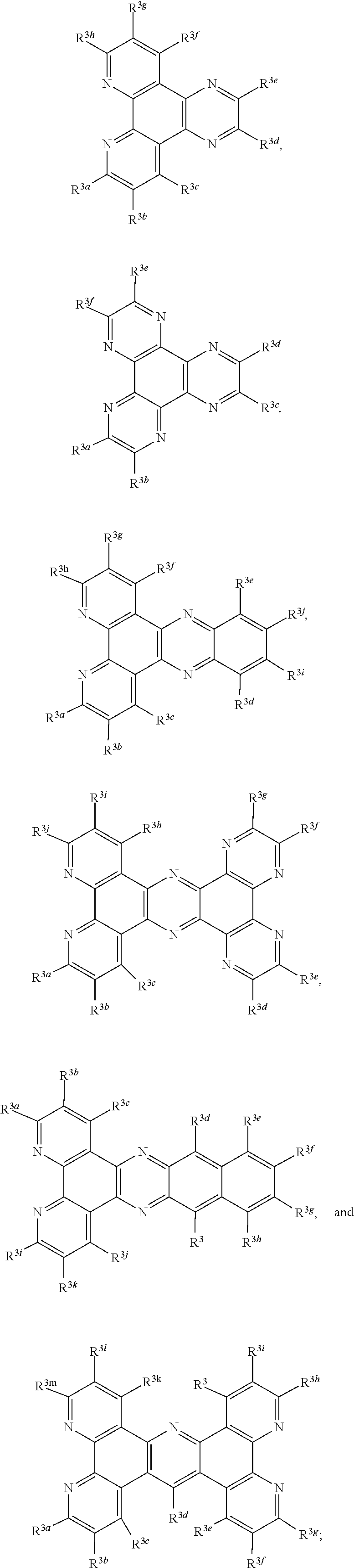 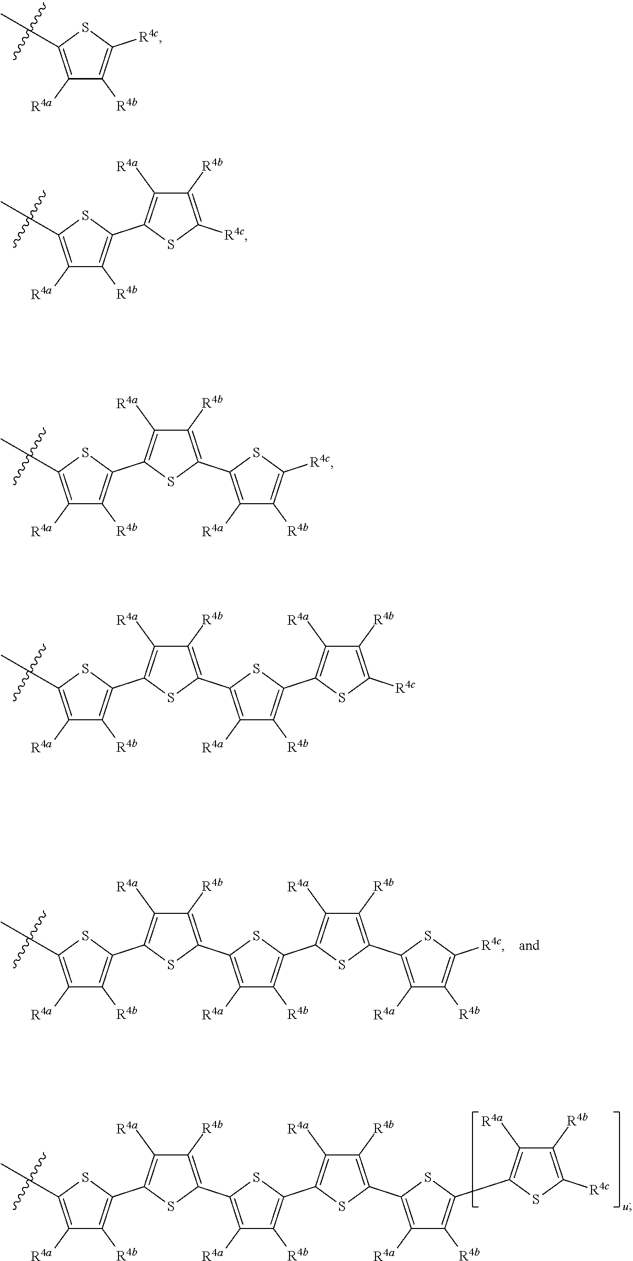
R¹ is selected from the group consisting of
u is an integer of 1 to 20;
R^{2a}, R^{2b}, R^{2c}, R^{2d}, R^{2e}, and R^{2f} at each occurrence are each independently selected from the group consisting of hydrogen, C1-6 optionally substituted alkyl, C1-6 optionally substituted branched alkyl, C3-7 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h} R³ⁱ, R^{3j}, R^{3k}, R^{3l}, and R^{3m} at each occurrence are each independently selected from the group consisting of hydrogen, C1-6 optionally substituted alkyl, C1-6 optionally substituted branched alkyl, C1-6 optionally substituted haloalkyl, C1-6 optionally substituted alkoxy, and CO₂R⁸;
R^{4a}, R^{4b}, and R^{4c} at each occurrence are each independently selected from the group consisting of hydrogen, C1-6 optionally substituted alkyl, C1-6 optionally substituted C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b}at each occurrence on a thiophene ring are taken together with the atom to which they are bound to form an optionally substituted ring having from 6 ring atoms containing 2 oxygen atoms;
R⁵ at each occurrence is independently selected from the group consisting of hydrogen and optionally substituted alkyl;
R⁶ at each occurrence is independently selected from the group consisting of hydrogen and optionally substituted alkyl;
R⁷ at each occurrence is independently selected from the group consisting of hydrogen and optionally substituted alkyl; and
R⁸ at each occurrence is independently selected from the group consisting of hydrogen and optionally substituted alkyl,
wherein:
the administering to the patient of the at least one metal complex is conducted prior to, during and/or after the administering to the patient of the at least one API; and
the at least one API has a structure not represented by Formula (I).

2. The method of claim 1, wherein the administering to the patient of the at least one metal complex causes an improvement in an efficacy of treating the condition relative to continued treatment of the condition with only the at least one API.

3. The method of claim 1, wherein the at least one metal complex is administered to the patient only after the condition has developed a resistance to the at least one API, which continues to be administered to the patient along with the at least one metal complex.

4. The method of claim 1, wherein the at least one metal complex is administered to the patient prior to or concurrently with the step of administering to the patient the at least one active pharmaceutical ingredient.

5. The method of claim 1, wherein the condition is cancer, and the at least one API is at least one member selected from the group consisting of a chemotherapeutic agent, an immunotherapeutic agent and a targeted therapeutic agent.

6. The method of claim 1, wherein the condition is an infectious disease and the at least one API is at least one member selected from the group consisting of an antibiotic agent, an antimicrobial agent, an antifungal agent and an antiviral agent.

7. The method of claim 1, wherein the at least one API is selected from the group consisting of Cisplatin, Temozolomide, Vandetanib, Withaferin A, Vemurafenib, Amiodarone, Vinblastine, Metformin, Gemcitabine and Acyclovir.

8. The method of claim 1, further comprising administering transferrin to the patient.

9. The method of claim 1, further comprising administering to the patient radiation selected from the group consisting of infrared light, visible light, X-rays and gamma rays.

10. The method of claim 1, wherein the at least one metal complex comprises at least one member selected from the group consisting of:
Ru(2,2′-bipyridine)₂(2-(2′,2″:5″,2″′-terthiophene)-imidazo[4,5-f][1,10] phenanthroline);
Ru(2,2′-bipyridine)₂(2-(2′,2″:5″,2″′;5″′,2″″-quaterthiophene)-imidazo[4,5-f][1,10] phenanthroline);
Ru(4,4′-dimethyl-2,2′-bipyridine)₂(2-thiophenimidazo[4,5-f][1,10] phenanthroline);
Ru(4,4′-dimethyl-2,2′-bipyridine)₂(2-(2′,2″-bithiophene)-imidazo[4,5-f][1,10] phenanthroline);
Ru(4,4′-dimethyl-2,2′-bipyridine)₂(2-(2′,2″:5″,2″′-terthiophene)-imidazo[4,5-f][1,10] phenanthroline);
Ru(4,4′-dimethyl-2,2′-bipyridine)₂(2-(2′,2″:5″,2″′;5″′,2″″-quaterthiophene) -imidazo[4,5-f][1,10] phenanthroline);
Ru(4,4′-di-t-butyl-2,2′-bipyridine)₂(2-thiophenimidazo[4,5-f][1,10] phenanthroline);
Ru(4,4′-di-t-butyl-2,2′-bipyridine)₂(2-(2′,2″-bithiophene)-imidazo[4,5-f][1,10] phenanthroline);
Ru(4,4′-di-t-butyl-2,2′-bipyridine)₂(2-(2′,2″:5″,2′″-terthiophene)-imidazo[4,5-f][1,10] phenanthroline);
Ru(4,4′-dimethoxy-2,2′-bipyridine)₂(2-(2′,2″:5″,2′″-terthiophene)-imidazo[4,5-f][1,10] phenanthroline);
Ru(5,5′-dimethyl-2,2′-bipyridine)₂(2-thiophenimidazo[4,5-f][1,10] phenanthroline);
Ru(5,5′-dimethyl-2,2′-bipyridine)₂(2-(2′,2″-bithiophene)-imidazo[4,5-f][1,10] phenanthroline);
Ru(5,5′-dimethyl-2,2′-bipyridine)₂(2-(2′,2″:5″,2′″-terthiophene)-imidazo[4,5-f][1,10] phenanthroline);
Ru(6,6′-dimethyl-2,2′-bipyridine)₂(2-thiophenimidazo[4,5-f][1,10] phenanthroline);
Ru(6,6′-dimethyl-2,2′-bipyridine)₂(2-(2′,2″-bithiophene)-imidazo[4,5-f][1,10] phenanthroline);
Ru(6,6′-dimethyl-2,2′-bipyridine)₂(2-(2′,2″:5″,2′″-terthiophene)-imidazo[4,5-f][1,10] phenanthroline);
Ru(4,4′-di (methylcarboxy)-2,2′-bipyridine)₂(2-(2′,2″:5″,2′″-terthiophene) -imidazo[4,5-f][1,10] phenanthroline);
Ru(2,2′-bipyrimidine)₂(2-(2′,2″:5″,2″″-terthiophene)-imidazo[4,5-f][1,10] phenanthroline);
Ru(2,2′-bipyrimidine)(4,4′-dimethyl-2,2′-bipyridine)(2-(2′,2″:5″,2″′-terthiophene)-imidazo[4,5-f][1,10] phenanthroline);
Ru(1,10-phenanthroline)₂(2-thiophenimidazo[4,5-f][1,10] phenanthroline);
Os(2,2′-bipyridine)₂(2-thiophenimidazo[4,5-f][1,10] phenanthroline);
Os(2,2′-bipyridine)₂(2-(2′,2″-bithiophene)-imidazo[4,5-f][1,10] phenanthroline);
Os(2,2′-bipyridine)₂(2-(2′,2″:5″,2′″-terthiophene)-imidazo[4,5-f][1,10] phenanthroline);
Os(1,10-phenanthroline)₂(2-thiophenimidazo[4,5-f][1,10] phenanthroline);
Os(1,10-phenanthroline)₂(2-(2′,2″-bithiophene)-imidazo[4,5-f][1,10] phenanthroline);
Os(1,10-phenanthroline)₂(2-(2′,2″:5″,2″′-terthiophene)-imidazo[4,5-f][1,10] phenanthroline);
Os(4,4′-dimethyl-2,2′-bipyridine)₂(2-thiophenimidazo[4,5-f][1,10] phenanthroline);
Os(4,4′-dimethyl-2,2′-bipyridine)₂(2-(2′,2″-bithiophene)-imidazo[4,5-f][1,10] phenanthroline);
Os(4,4′-dimethyl-2,2′-bipyridine)₂(2-(2′,2″:5″,2′″-terthiophene)-imidazo[4,5-f][1,10] phenanthroline); and
pharmaceutically acceptable salts thereof.

11. The method of claim 1, wherein the at least one metal complex is Ru(4,4′-dimethyl-2,2′-bipyridine)₂(2-(2′,2″:5″,2′-terthiophene)-imidazo[4,5-f][1,10] phenanthroline) or a pharmaceutically acceptable salt thereof.

12. A composition for combination therapy effective to treat a condition in a patient, said composition comprising:
at least one active pharmaceutical ingredient (API) selected from the group consisting of Cisplatin, Temozolomide, Vandetanib, Withaferin A, Vemurafenib, Amiodarone, Vinblastine, Metformin, Gemcitabine and Acyclovir; and
at least one metal complex effective to potentiate an efficacy of the active pharmaceutical ingredient to treat the condition, wherein the at least one metal complex is represented by formula (I): 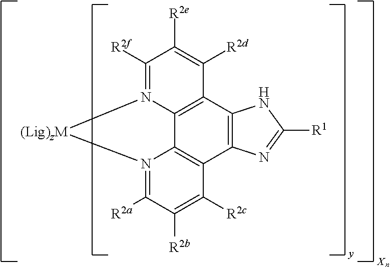
including hydrates, solvates, pharmaceutically acceptable salts and prodrugs thereof, wherein:
M is selected from the group consisting of manganese, molybdenum, rhenium, iron, ruthenium, osmium, cobalt, rhodium, iridium, nickel, platinum, and copper;
X is selected from the group consisting of Cl⁻, PF₆⁻, Br⁻, BF₄⁻, ClO₄⁻, CF₃SO₃⁻, and SO₄⁻²;
n=0, 1, 2, 3, 4, or 5;
y =1, 2, or 3;
z=0, 1, or 2;
Lig at each occurrence is independently selected from the group consisting of 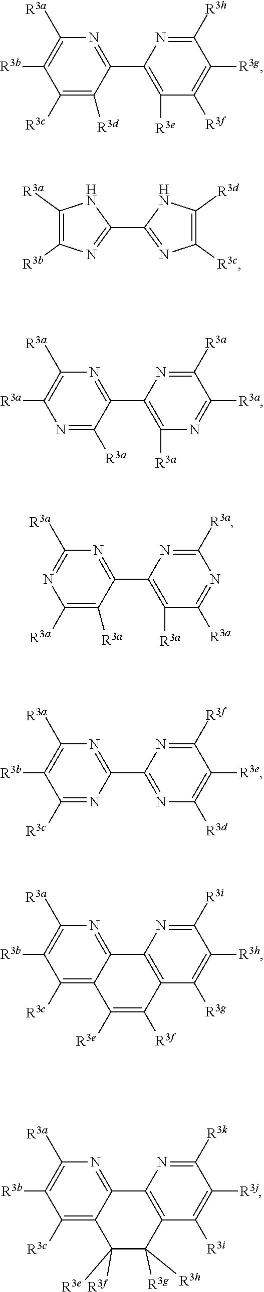 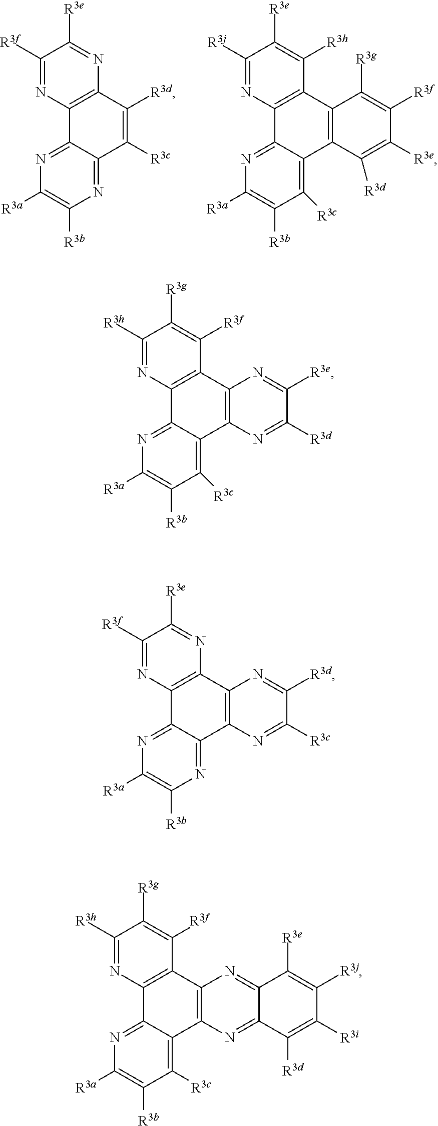 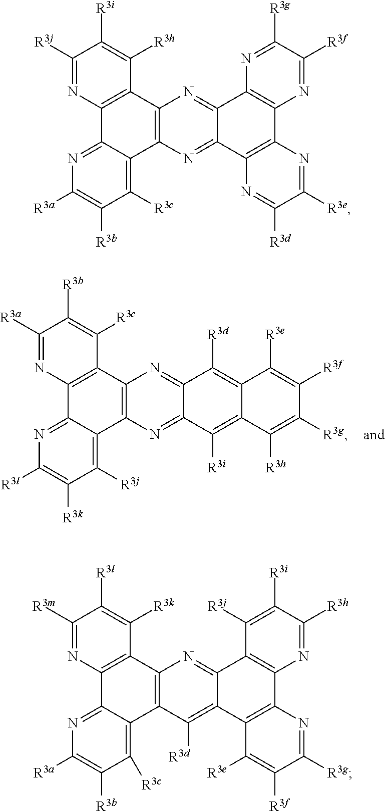 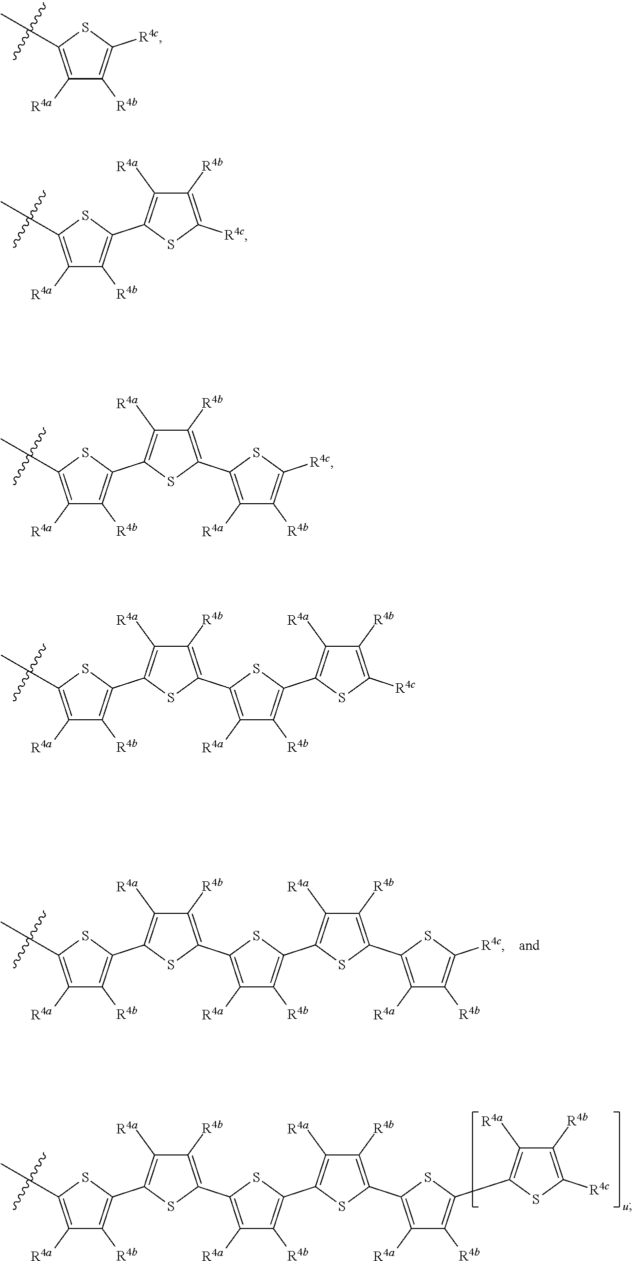
R¹ is selected from the group consisting of
u is an integer of 1 to 20;
R^{2a}, R^{2b}, R^{2c}, R^{2d}, R^{2e}, and R^{2f} at each occurrence are each independently selected from the group consisting of hydrogen, C1-6 optionally substituted alkyl, C1-6 optionally substituted branched alkyl, C3-7 optionally substituted cycloalkyl, C1-6 optionally substituted haloalkyl, C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{3a}, R^{3b}, R^{3c}, R^{3d}, R^{3e}, R^{3f}, R^{3g}, R^{3h} R³ⁱ, R^{3j}, R^{3k}, R^{3l}, and R^{3m} at each occurrence are each independently selected from the group consisting of hydrogen, C1-6 optionally substituted alkyl, C1-6 optionally substituted branched alkyl, C1-6 optionally substituted haloalkyl, C1-6 optionally substituted alkoxy, and CO₂R⁸;
R^{4a}, R^{4b}, and R^{4c} at each occurrence are each independently selected from the group consisting of hydrogen, C1-6 optionally substituted alkyl, C1-6 optionally substituted C1-6 optionally substituted alkoxy, CO₂R⁵, CONR⁶₂, NR⁷₂, sulfate, sulfonate, optionally substituted aryl, optionally substituted aryloxy, optionally substituted heteroaryl, and optionally substituted heterocycle;
R^{4a} and R^{4b} at each occurrence on a thiophene ring are taken together with the atom to which they are bound to form an optionally substituted ring having from 6 ring atoms containing 2 oxygen atoms;
R⁵ at each occurrence is independently selected from the group consisting of hydrogen and optionally substituted alkyl;
R⁶ at each occurrence is independently selected from the group consisting of hydrogen and optionally substituted alkyl;
R⁷ at each occurrence is independently selected from the group consisting of hydrogen and optionally substituted alkyl; and
R⁸ at each occurrence is independently selected from the group consisting of hydrogen and optionally substituted alkyl.

13. The composition of claim 12, wherein the condition is cancer.

14. The composition of claim 12, wherein the condition is an infectious disease.

15. The composition of claim 12, further comprising transferrin.

16. The composition of claim 12, wherein the at least one metal complex comprises at least one member selected from the group consisting of:
Ru(2,2′-bipyridine)₂(2-(2′,2″:5″,2″′-terthiophene)-imidazo[4,5-f][1,10] phenanthroline);
Ru(2,2′-bipyridine)₂(2-(2′,2″:5″,2″′;5″′,2″″-quaterthiophene)-imidazo[4,5-f][1,10] phenanthroline);
Ru(4,4′-dimethyl-2,2′-bipyridine)₂(2-thiophenimidazo[4,5-f][1,10] phenanthroline);
Ru(4,4′-dimethyl-2,2′-bipyridine)₂(2-(2′,2″-bithiophene)-imidazo[4,5-f][1,10] phenanthroline);
Ru(4,4′-dimethyl-2,2′-bipyridine)₂(2-(2′,2″:5″,2′″-terthiophene)-imidazo[4,5-f][1,10] phenanthroline);
Ru(4,4′-dimethyl-2,2′-bipyridine)₂(2-(2′,2″:5″,2″′;5′″,2″″-quaterthiophene) -imidazo[4,5-f][1,10] phenanthroline);
Ru(4,4′-di-t-butyl-2,2′-bipyridine)₂(2-thiophenimidazo[4,5-f][1,10] phenanthroline);
Ru(4,4′-di-t-butyl-2,2′-bipyridine)₂(2-(2′,2″-bithiophene)-imidazo[4,5-f][1,10] phenanthroline);
Ru(4,4′-di-t-butyl-2,2′-bipyridine)₂(2-(2′,2″:5″,2′″-terthiophene)-imidazo[4,5-f][1,10] phenanthroline);
Ru(4,4′-dimethoxy-2,2′-bipyridine)₂(2-(2′,2″:5″,2″′-terthiophene)-imidazo[4,5-f][1,10] phenanthroline);
Ru(5,5′-dimethyl-2,2′-bipyridine)₂(2-thiophenimidazo[4,5-f][1,10] phenanthroline);
Ru(5,5′-dimethyl-2,2′-bipyridine)₂(2-(2′,2″-bithiophene)-imidazo[4,5-f][1,10] phenanthroline);
Ru(5,5′-dimethyl-2,2′-bipyridine)₂(2-(2′,2″:5″,2′″-terthiophene)-imidazo[4,5-f][1,10] phenanthroline);
Ru(6,6′-dimethyl-2,2′-bipyridine)₂(2-thiophenimidazo[4,5-f][1,10] phenanthroline);
Ru(6,6′-dimethyl-2,2′-bipyridine)₂(2-(2′,2″-bithiophene)-imidazo[4,5-f][1,10] phenanthroline);
Ru(6,6′-dimethyl-2,2′-bipyridine)₂(2-(2′,2″:5″,2′″-terthiophene)-imidazo[4,5-f][1,10] phenanthroline);
Ru(4,4′-di (methylcarboxy)-2,2′-bipyridine)₂(2-(2′,2″:5″,2′″-terthiophene) -imidazo[4,5-f][1,10] phenanthroline);
Ru(2,2′-bipyrimidine)₂(2-(2′,2″:5″,2″′-terthiophene)-imidazo[4,5-f][1,10] phenanthroline);
Ru(2,2′-bipyrimidine)(4,4′-dimethyl-2,2′-bipyridine)(2-(2′,2″:5″,2″′-terthiophene)-imidazo[4,5-f][1,10] phenanthroline);
Ru(1,10-phenanthroline)₂(2-thiophenimidazo[4,5-f][1,10] phenanthroline);
Os(2,2′-bipyridine)₂(2-thiophenimidazo[4,5-f][1,10] phenanthroline);
Os(2,2′-bipyridine)₂(2-(2′,2″-bithiophene)-imidazo[4,5-f][1,10] phenanthroline);
Os(2,2′-bipyridine)₂(2-(2′,2″:5″,2′″-terthiophene)-imidazo[4,5-f][1,10] phenanthroline);
Os(1,10-phenanthroline)₂(2-thiophenimidazo[4,5-f][1,10] phenanthroline);
Os(1,10-phenanthroline)₂(2-(2′,2″-bithiophene)-imidazo[4,5-f][1,10] phenanthroline);
Os(1,10-phenanthroline)₂(2-(2′,2″:5″,2″′-terthiophene)-imidazo[4,5-f][1,10] phenanthroline);
Os(4,4′-dimethyl-2,2′-bipyridine)₂(2-thiophenimidazo[4,5-f][1,10] phenanthroline);
Os(4,4′-dimethyl-2,2′-bipyridine)₂(2-(2′,2″-bithiophene)-imidazo[4,5-f][1,10] phenanthroline);
Os(4,4′-dimethyl-2,2′-bipyridine)₂(2-(2′,2″:5″,2″-terthiophene)-imidazo[4,5-f][1,10] phenanthroline); and
pharmaceutically acceptable salts thereof.

17. The composition of claim 12, wherein the at least one metal complex is
Ru(4,4′-dimethyl-2,2′-bipyridine)₂(2-(2′,2″:5″,2″-terthiophene)-imidazo[4,5-f][1,10] phenanthroline); ) or a pharmaceutically acceptable salt thereof.
